(19) United States
(12) Patent Application Publication
(10) Pub. No.: US 2025/0369876 A1
(43) Pub. Date: Dec. 4, 2025

(54) BREATH ANALYSIS WITH CAVITY-ENHANCED DIRECT FREQUENCY-COMB SPECTROSCOPY

(71) Applicants: THE REGENTS OF THE UNIVERSITY OF COLORADO, A BODY CORPORATE, Denver, CO (US); GOVERNMENT OF THE UNITED STATES OF AMERICA, AS REPRESENTED BY THE SECRETARY OF COMMERCE, Gaithersburg, MD (US)

(72) Inventors: Jun YE, Louisville, CO (US); Qizhong LIANG, Boulder, CO (US); Jutta TOSCANO, Basel (CH); Ya-Chu CHAN, Boulder, CO (US); David J. NESBITT, Boulder, CO (US)

(21) Appl. No.: 18/877,909

(22) PCT Filed: Jun. 22, 2023
(86) PCT No.: PCT/US2023/026020

§ 371 Date: Dec. 20, 2024

Related U.S. Application Data
(60) Provisional application No. 63/366,779, filed on Jun. 22, 2022.

(51) Int. Cl. G01N 21/31; G01N 33/497; G16H 50/20

(52) U.S. Cl. CPC G01N 21/3103; G01N 33/4975; G16H 50/20

(57) ABSTRACT

A method for analyzing a system includes performing cavity-enhanced direct frequency-comb spectroscopy to obtain a measured absorption spectrum that indicates transmission of an optical frequency comb through a sample derived from the system. The method includes feeding the measured absorption spectrum into a trained machine-learning model to generate a model output. The machine-learning model may be trained to perform classification, in which case the model output may include a prediction that the system is in a particular state. The machine-learning model may also be trained to perform regression, in which case the model output may include a test score indicating the severity of a particular state of the system. In some embodiments, the system is a human subject and the sample is breath obtained non-invasively from the subject. In these embodiments, the model output may indicate whether the subject has an infection, illness, or physical condition.

### RELATED APPLICATIONS

This application claims priority to U.S. Provisional Patent Application No. 63/366,779, filed on Jun. 22, 2022, the entirety of which is incorporated herein by reference.

### STATEMENT REGARDING FEDERALLY SPONSORED RESEARCH OR DEVELOPMENT

This invention was made with government support under grant number 9FA9550-19-1-0148 awarded by the Air Force Office of Scientific Research. The government has certain rights in the invention.

### BACKGROUND

A biomarker is a measurable indicator of a disease or physical condition in an organism. The physical condition may be a normal biological process, a pathogenic process, or a response to a therapeutic intervention (e.g., a pharmacological response to a prescribed medication). For clinical purposes, biomarkers may be used to guide or narrow treatment options for a patient. More specifically, biomarkers may be used predictively (i.e., to predict clinical outcomes for the patient), diagnostically (i.e., to help diagnose the patient), or prognostically (i.e., to identify overall outcomes).

### SUMMARY

The spread of the SARS-CoV-2 (severe acute respiratory syndrome coronavirus-2) has renewed interest in improving testing that can detect the COVID-19 disease state, and others. Currently, the most accurate diagnosis of SARS-Cov-2 uses polymerase chain reaction (PCR), such as quantitative reverse transcription PCR (RT-qPCR), which amplifies DNA and RNA sequences to make them easier to detect. Nasal swab tests using PCR-based detection are accurate, but have several limitations, including how the samples are handled (e.g., improper swabbing and storage), the requirement that sampling occurs during the acute phase, and a long testing time. For example, it can take 2 to 4 hours for PCR acquisition, and more than 12 hours for overall processing and handling. PCR machines are also large, expensive, and require technicians to operate properly.

Antigen tests are also now commonly used to detect SARS-CoV-2. Antigen tests identify the presence of a virus in nose and throat secretions by looking for proteins made by the virus (as opposed to directly detecting the genetic material). Advantageously, antigen tests take only 15 minutes, are inexpensive, and can be performed at-home without a medical professional or expensive equipment. However, antigen tests do not have the accuracy of PCR-based tests and are known for high rates of false negatives, especially for patients with a low viral load. Antigen tests may also give incorrect results due to improper handling (e.g., insufficient swabbing). They also require reagents, which can be difficult to produce and obtain in the middle of a pandemic.

More recently, light-based diagnosis techniques are being explored to combine the sensitivity and specificity of PCR-based testing with the low cost, high-speed, and scalability of antigen tests. Some of these light-based tests do not require reagents, thereby eliminating an important problem with PCR and antigen-based tests. These light-based tests perform spectroscopy (e.g., attenuated total reflection Fourier-transform infrared (ATR-FTIR) spectroscopy) on a sample obtained from a nasal swab or gargle to identify a spectral signature that is known to correlate with the presence of COVID-19.

The present disclosure includes embodiments that use cavity-enhanced direct frequency-comb spectroscopy (CE-DFCS) to obtain a measured absorption spectrum of a gas sample obtained from a system (e.g., a human subject). In some embodiments, the gas is a sample of exhaled breath that is obtained non-invasively from a human subject (as opposed to an invasive nasal swab). Advantageously, CE-DFCS offers greater measurement sensitivity to gaseous molecular species than the prior-art techniques described above, and therefore has the potential to improve predictive and diagnostic accuracy. In particular, when CE-DFCS is implemented in the mid-infrared (i.e., approximately 3-8 μm), frequency-comb light interacts with the fundamental vibrational resonances of many molecular species in the gas, which generate stronger absorption signals than higher-order overtones at shorter wavelengths.

The measured absorption spectrum is fed into a machine-learning model that was previously trained using a supervisory set of CE-DFCS spectra. The machine-learning model outputs a prediction that indicates whether or not the system is in a particular state (e.g., whether or not a human subject has COVID-19 or not). Alternatively or additionally, the machine-learning model outputs a quantitative indication of the severity or intensity of a particular state or condition of the system.

One aspect of the present embodiments is the discovery that COVID-19 affects the molecular makeup of human breath, and that therefore spectroscopy of human breath can be used as a diagnostic tool to identify COVID-19. The machine-learning analysis of the present embodiments is tailored to the detection principle of CE-DFCS. CE-DFCS utilizes both the evenly spaced, isolated nature of the light emitted from a frequency comb and a high-resolution spectrometer capable of resolving individual comb lines to realize spectroscopy data collection down to frequency uncertainties specified by the linewidth of each comb teeth and at a well-defined frequency sampling interval specified by the spacing of adjacent comb lines. The highly reliable frequency axis provided by CE-DFCS separates it from other broadband absorption spectroscopy techniques and ensures the chemical information presented over the spectral range can be collected in a most extensive manner. The measured spectrum may contain thousands of data points, or more, each carrying chemical information at a well-defined optical frequency.

Mid-infrared CE-DFCS advantageously offers sensitivities at the parts-per-trillion level. As a result, CE-DFCS can detect hundreds to thousands, or more, of molecular species present in the sample. Because currently available molecular cross-section databases allow only a few tens of molecules to be simultaneously fitted to theoretical absorption curves, the richness of the chemical information collected by CE-DFCS requires a tailored, pattern-based way of machine-learning analysis that is described herein. In traditional techniques, the lack of chemical information (which typically arises from insufficient detection sensitivity) usually can be paired well with fitting the spectrum with a molecular cross-sectional database and using the fitted concentrations for subsequent machine-learning analysis. Such traditional techniques are referred to herein as “species-based.”

In the present embodiments, signals obtained from CE-DFCS spectra are used directly as predictor variables for machine-learning analysis. This approach is referred to herein as “pattern-based.” Advantageously, pattern-based analysis of CE-DFCS spectra ensures that all chemical information in the spectra is utilized for making predictions with the highest possible accuracy. As described in more detail below, a real-world clinical study has confirmed that such analysis leads to better prediction performance and confirms the extra richness incapable to be utilized by the species-based approach can be better utilized by the pattern-based approach.

### BRIEF DESCRIPTION OF THE FIGURES

FIG. 1 is a functional diagram of an apparatus for analyzing a gas using cavity-enhanced direct frequency-comb spectroscopy (CE-DFCS), in embodiments.

FIG. 2 shows an artificial neural network that is one example of a machine-learning model, in an embodiment.

FIG. 3 is a functional diagram of a computational device that is one example of a signal processor, in an embodiment.

FIG. 4 shows a CE-DFCS breathalyzer, in an embodiment. Panel (a) shows a schematic representation of the working principle of the device. An exhaled human breath sample was collected in a Tedlar bag and then loaded into an analysis chamber. The chamber was surrounded by a pair of high-reflectivity optical mirrors. A mid-infrared frequency comb laser interacted with the loaded sample and generated a broadband molecular absorption spectrum. The spectroscopy data was then used for supervised machine learning analysis to predict the binary response class for the research subject (either positive or negative). Panel (b) shows an example of an absorption spectrum of a sample collected from a research subject's exhaled breath (top). Inverted in sign and plotted with different shading are four fitted species (CH₃OH, H₂O, HDO, and CH₄) that give the most dominant absorption features.

FIG. 5 is a plot showing the number of COVID-19 symptoms experienced by the positive participants. Only SARS-CoV-2 positive participants with non-missing questionnaire responses were included.

FIG. 6 illustrates prediction performance for SARS-CoV-2 infection. Panels (a)-(c) and panels (d)-(f) show prediction results obtained by the pattern-based approach and the molecule-based approach, respectively. A control based on birth month (panels (a) and (d)) examines whether subjects were born on the even or the odd months. A control based on breath vs. ambient air (panels (c) and (f)) examines whether spectroscopy data were measured for inhaled air or exhaled breath. Obtained areas under the curve (AUCs) are reported in the panels. Respective assignment of the response classes for the two controls to positive and negative was done at random and does not carry any particular meaning. In FIG. 6, “TP” means true positive while “FP” means false positive.

FIG. 7 illustrates the pattern-based approach over the molecule-based approach. In panels (a) and (b), distribution of the subjects' data for the first three partial least squares (PLS) components, with down-pointing and up-pointing triangles representing positive and negative research subjects, respectively. In panels (c) and (d), variable importance in the projection (VIP) scores show the importance of different predictor variables in prediction making. Predictor variables with VIP scores above (or below) unity were considered as important (or unimportant) for predictions. Results shown for the pattern-based (panels (a) and (c)) and molecule-based (panels (c) and (d)) approaches were calculated using the complete data set (N=170) for SARS-CoV-2 infection.

FIG. 8 illustrates prediction performance for a list of potential confounders. As shown in panels (a)-(c), random guessing results (AUC<0.6) were found for alcohol use, age, and lactose intolerance, respectively. As shown in panels (d)-(g), significant differences (0.6≤AUC<0.7) were found for smoking, abdominal pain, sex, and constipation, respectively. Class assignments for each response type are shown in the figure. For age, a median age of 23 years old was used for class assignment. All results shown were analyzed by the pattern-based approach.

FIG. 9 illustrates the total percentage variance explained in the response. Panels (a) and (b) show results for the molecule-based approach and pattern-based approach, respectively.

FIG. 10 shows the AUC calculated for different numbers of PLS components and different training and testing set partition ratios. For different partition ratios, we show the testing set size in plotting the results. The training set size can be obtained by subtracting the testing set size from the complete data set size (N=170). Panels (a)-(c) show results for the molecule-based approach, for birth month, sex, and SARS-CoV-2, respectively. Panels (d)-(f) show results for the pattern-based approach, for birth month, sex, and SARS-CoV-2, respectively.

### DETAILED DESCRIPTION

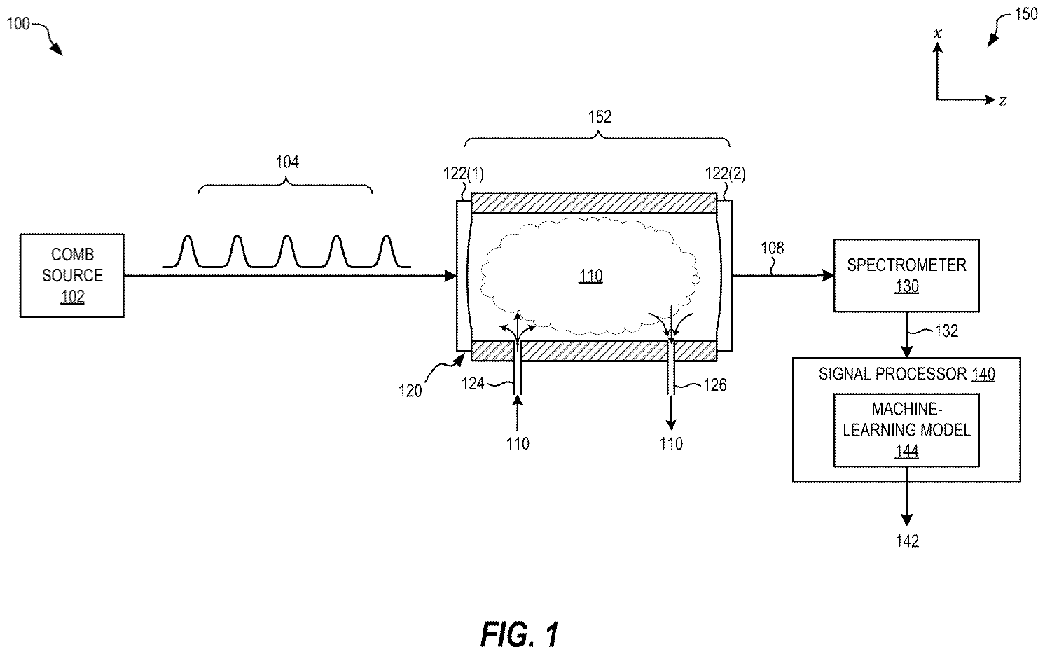

FIG. 1 is a functional diagram of an apparatus 100 for analyzing a sample obtained from a system. In the example of FIG. 1, the sample is a gas 110 that is measured using cavity-enhanced direct frequency-comb spectroscopy (CE-DFCS). The gas 110 is confined within a cell 120 that is axially bounded along z (see right-handed coordinate system 150) by a first mirror 122(1) and a second mirror 122(2) that counterface each other to create an optical cavity 152. The optical cavity 152 may be confocal, half-confocal, plane-parallel (i.e., Fabry-Perot), or another configuration known in the art. The number, type, and quantity of constituents in the gas 110 affect the measured spectrum, from which information is derived about a state or condition of the system from which the gas 110 was obtained or derived.

The gas 110 may be introduced into the cell 120, and therefore the optical cavity 152, via an input port 124. Similarly, the gas 110 may be evacuated from the cell 120 via an output port 126. Thus, the ports 124 and 126 allow the gas 110 to continuously flow through the cell 120 while it is being measured. Alternatively, a valve may be located on one or both of the ports 124 and 126 to allow the gas 110 to be confined, without flow, inside the cell 120 while it is being measured. For example, while the valve on the output port 126 is closed, gas 110 may flow into the cell 120 until a setpoint pressure is reached, at which point the valve on the input port 124 may then be closed. The gas 110 inside the cell 120 may then be measured at the setpoint pressure.

To implement CE-DFCS with the apparatus 100, an optical frequency comb 104 is transmitted through the first mirror 122(1) to excite longitudinal modes of the optical cavity 152. In some embodiments, the apparatus 100 includes a comb source 102 operable to generate the optical frequency comb 104. The apparatus 100 may also include optics for steering and mode-matching the optical frequency comb 104 to the optical cavity 152. In FIG. 1, the optical frequency comb 104 is illustrated as a pulse train of optical pulses. In this case, the comb source 102 may be a femtosecond pulsed laser (e.g., Ti:Saph, fiber, diode, etc.). Other techniques or photonic devices may be used to generate the optical frequency comb 104.

Although not shown in FIG. 1, the optical frequency comb 104 has a comb-like spectrum formed from a series of discrete frequency components, or teeth, that are equally separated in frequency by a repetition rate fᵣ of the comb source 102. The spectrum may cover any region of the electromagnetic spectrum (e.g., ultraviolet, visible, infrared, etc.). If the comb-like spectrum were to extend to zero frequency, the tooth closest to zero would be shifted from zero by a comb-offset frequency f₀. The optical frequency comb 104 may have up to tens of thousands of teeth, or more, spanning up to hundreds of nanometers, or more.

Techniques known in the art may be used to frequency-stabilize the teeth of the optical frequency comb 104. In the case of FIG. 1, the frequencies may be stabilized to the longitudinal resonances of the optical cavity 152 by controlling the free-spectral range of the optical cavity 152 to equal the repetition rate fᵣ (or vice versa) or an integer multiple thereof. Due to dispersion of the mirrors 122(1) and 122(2), the free-spectral range of the optical cavity 152 may not be uniform across the full spectrum of the optical frequency comb 104. Accordingly, it may only be possible for a portion of the optical frequency comb 104 (i.e., a subset of the frequency components) to be simultaneously resonant with the optical cavity 152. One or both of the repetition rate fᵣ and comb-offset frequency f₀ may be controlled to change the bandwidth of the portion of the optical frequency comb 104 that is resonant with the optical cavity 152.

The apparatus 100 also includes a spectrometer 130 that measures an amplitude or power of each tooth of an output beam 108. Some of the light that is resonant inside the optical cavity 152 passes through the second mirror 122(2) to form the output beam 108, which has the same comb-like structure as the optical frequency comb 104. However, due to absorption by the gas 110, some of the teeth of the output beam 108 have less power than their corresponding teeth of the optical frequency comb 104. The spectrometer 130 outputs an absorption spectrum 132, which may be a vector or an array whose elements quantify the absorbed power of the teeth or the transmission of the teeth through the gas 110 and optical cavity 152. In this case, the array index may be used to identify the frequency or wavelength of the corresponding tooth.

The apparatus 100 also includes a signal processor 140 that processes the absorption spectrum 132 by feeding it into a machine-learning model 144. The machine-learning model 144 has been previously trained with a supervisory set of CE-DFCS spectra. For example, the supervisory set may include CE-DFCS spectra obtained from gas samples having known constituents and quantities, and therefore known absorption spectra. Alternatively or additionally, the supervisory set may include CE-DFCS spectra measured from a sampled system within a known state or condition (e.g., a human patient that does or does not have Covid-19). Supervisory CE-DFCS spectra may be measured experimentally or calculated theoretically (e.g., the output of a numerical simulation).

The machine-learning model 144 processes the absorption spectrum 132 to generate a model output 142. The model output 142 may include a binary-valued prediction of whether or not the system is in one particular state (e.g., “infected” or “not infected”). Alternatively or additionally, the model output 142 may include a multi-valued prediction indicating which one of a plurality of states the system is in. For example, the plurality of states may include one or more of a disease state, a non-disease state, a physiological state, a chemical state, a medical state, and a functional state. The disease state may indicate the presence of an infection caused by a pathogen (e.g., SARS-CoV-2). in a human subject. Alternatively or additionally, the model output 142 may include a continuous-valued test score that quantitatively indicates the severity or intensity of a particular state of the system. For example, the test score may indicate the severity of an infection caused by a pathogen (e.g., SARS-CoV-2) in a human subject.

In some embodiments, the sampled system is biological, such as an organism (e.g., human being, animal, microorganism, etc.) or natural ecosystem. For example, the gas 110 may be a breath sample exhaled by a human subject. In this case, the human subject may exhale into a storage vessel (e.g., a polyvinyl fluoride bag) that stores the breath sample prior to flowing into the cell 120. In this case, the gas 110 is obtained from the sampled system directly, i.e., without additional processing. Alternatively, the gas 110 may be obtained indirectly, i.e., by processing a gas, liquid, or solid sample directly obtained from the sampled system. For example, the sample may be heated to vaporize at least part of it into the gas 110. Alternatively, the sample may be chemically treated to create a chemical reaction that generates the gas 110.

In other embodiments, the sampled system is not biological. Examples include manufacturing facilities, furnaces, water treatment facilities, natural-gas infrastructure (e.g., tanked, pipelines, wells, condensation facilities, etc.), oil refineries, chemical plants, vehicles, and so on. The sampled system may be another type of non-biological system without departing from the scope hereof. In all these examples, the sampled system emits gases, liquids, or solids (or a combination thereof) that can be analyzed, either directly or after processing, to determine what state the system is in or to derive information about the state of the system.

In embodiments, a subject (human or animal) may be diagnosed based on the model output 142. For example, the subject may be diagnosed as having a disease or medical condition, as predicted and indicated by the model output 142. The subject may be further provided with one or more therapeutic interventions for treating the disease or medical condition. Examples of such therapeutic interventions include, but are not limited to, surgical procedures, non-surgical medical procedures, and prescriptions for one or more pharmaceutical drugs.

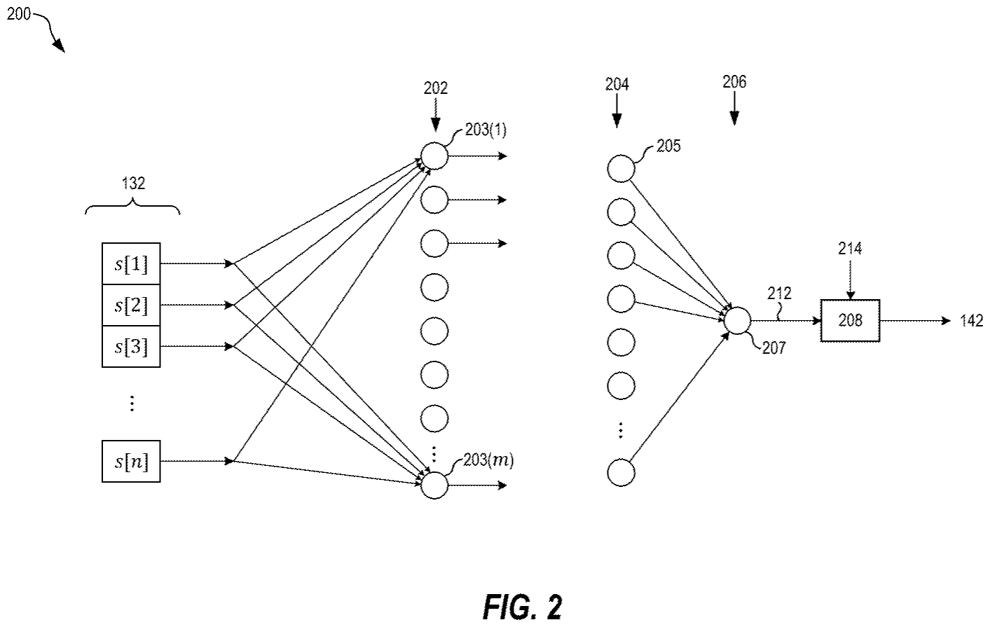

FIG. 2 shows an artificial neural network (ANN) 200 that is one example of the machine-learning model 144 of FIG. 1. In FIG. 2, nodes of the ANN 200 are indicated by circles and weights are indicated by lines connected thereto. The ANN 200 includes a plurality of m input nodes 203(1) . . . 203(m) that form an input layer 202. The ANN 200 also includes internal nodes 205 forming one or more hidden layers 204. For clarity in FIG. 2, only one of the internal nodes 205 is labeled. The ANN 200 also includes one or more output nodes 207 forming an output layer 206. In the example of FIG. 2, the output layer 206 contains only one output node 207 that outputs one output value 212. In other embodiments, the output layer 206 contains more than one output node 207, in which case the ANN 200 outputs more than one output value 212. The nodes 203, 205, and 207 may have any combination of offsets and activation functions known in the art.

The absorption spectrum 132 is fed into the input layer 202. The absorption spectrum 132 is represented in FIG. 2 as an array s indexed 1 to n. Each element s[i] of the array stores an absorption value for a corresponding tooth of the optical frequency comb 104. The number n of elements may be as high as several thousand, or more. In FIG. 1, each element s[i] is fully connected to the input nodes 203(1) . . . 203(m). Alternatively, each element s[i] is sparsely connected to the input nodes 203(1) . . . 203(m). For example, in one embodiment, each element s[i] is only connected to a corresponding one of the input nodes 203(i). In this embodiment, the number m of input nodes 203 equals the number n of elements. Similarly, the hidden layers 204 may be fully connected, sparsely connected, or a combination thereof.

The ANN 200 may include or incorporate one or more other neural-network architectures/features known in the art. Examples include max-pooling layers, convolution layers, and recurrent layers. The signal processor 140 may pre-process the absorption spectrum 132 before feeding it into the input layer 202. Additionally or alternatively, the signal processor 140 may post-process the output value 212 to transform it into the model output 142. In one example of post-processing, the output value 212 is fed into a threshold detector 208 that outputs a binary value based on whether the output value 212 is greater than or less than a threshold 214. This binary value may form part or all of the model output 142.

In other embodiments, the machine-learning model 144 is not a neural network. Examples include support-vector machines, decision trees, regression analysis, Bayesian networks, and genetic algorithms. It should also be understand that the machine-learning model 144 may be a plurality of machine-learning models, each trained differently (e.g., to perform different tasks). In this case, the absorption spectrum 132 may be fed, in parallel, to the plurality of machine-learning models to generate a respective plurality of model outputs. These outputs may be aggregated to generate the model output 142.

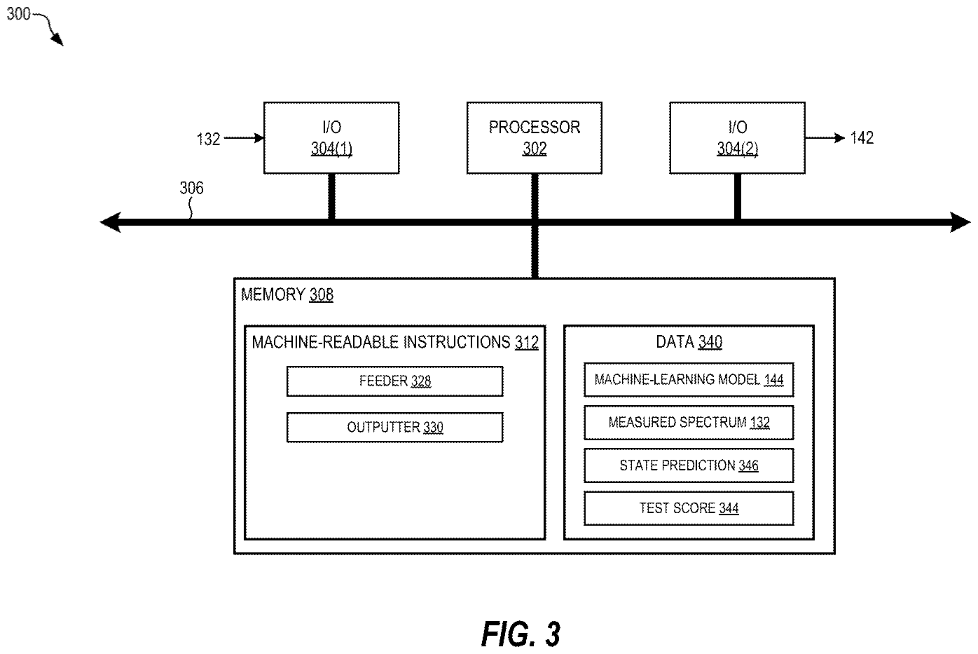

FIG. 3 is a functional diagram of a computational device 300 that is one example of the signal processor 140 of FIG. 1. The computational device 300 may be implemented, for example, as an embedded system co-located with other components of the apparatus 100. Alternatively, the computational device 300 may be remote from the other components of the apparatus 100. The computational device 300 includes a memory 308 that communicates with a processor 302 over a system bus 306. In some embodiments, the computational device 300 also includes a graphical display (not shown) for visually displaying information to a user, receiving input from the user, or both. Alternatively, the computational device 300 may include a display adapter for use with a graphical display provided by a third party.

The computational device 300 also includes a first input/output (I/O) block 304(1) that interfaces with the spectrometer 130 to receive the measured spectrum 132. The computational device 300 also includes a second I/O block 304(2) through which it may communicate with a peripheral device or remote computer system (e.g., hard drive, USB port, memory card, network connector, etc.). For example, the computational device 300 may output the model output 142 as data via the I/O block 304(2). The I/O blocks 304(1) and 304(2) are also connected to the system bus 306 and therefore can communicate with the processor 302, store data in the memory 308, and retrieve data from the memory 308.

The processor 302 may be any type of circuit capable of performing logic, control, and input/output operations. For example, the processor 302 may include one or more of a microprocessor with one or more central processing unit (CPU) cores, a graphics processing unit (GPU), a digital signal processor (DSP), a field-programmable gate array (FPGA), a system-on-chip (SoC), and a microcontroller unit (MCU). The processor 302 may also include a memory controller, bus controller, one or more co-processors, and/or other components that manage data flow between the processor 302 and other components communicably coupled to the system bus 306. The processor 302 may be implemented as a single integrated circuit (IC), or as a plurality of ICs. In some embodiments, one or more of the processor 302, memory 308, I/O block 304(1), and I/O block 304(2) are implemented as a single IC. The processor 302 may use a complex instruction set computing (CISC) architecture, or a reduced instruction set computing (RISC) architecture.

The memory 308 stores machine-readable instructions 312 that, when executed by the processor 302, control the computational device 300 to implement the functionality of the signal processor 140, as described herein. The memory 308 also stores data 340 used by the processor 302 when executing the machine-readable instructions 312. In the example of FIG. 3, the data 340 includes the machine-learning model 144, the measured spectrum 132, a state prediction 346, and a test score 344. The state prediction 346 and test score 344 may be thought of as the model output 142 of FIG. 1. The machine-readable instructions 312 include a feeder 328 that feeds the measured spectrum 132 into the machine-learning model 144 and executes the machine-learning model 144 to generate the state prediction 346, test score 344, or both. The machine-readable instructions 312 also include an outputter 330 that outputs one or both of the state prediction 346 and test score 344. The memory 308 may store additional machine-readable instructions 312 than shown without departing from the scope hereof. Similarly, the memory 308 may store additional data 340 than shown without departing from the scope hereof.

In some embodiments, the processor 302 does not execute machine-readable instructions (e.g., an FPGA) to implement the functionality described here. Rather, the processor 302 is pre-programmed to perform tasks and therefore acts like a hard-wired circuit. Accordingly, in these embodiments the functionality is implemented only in hardware and the machine-readable instructions 312 may be excluded. In other embodiments, such as shown in FIG. 3, the functionality is implemented only in software. In yet other embodiments, this functionality is implemented as a combination of hardware and software.

While FIG. 3 shows the computational device 300 with one system bus 306, the computational device 300 may be implemented with a different type of architecture without departing from the scope hereof. For example, the machine-readable instructions 312 and data 340 may be stored in separate memories that communicate with the processor 1004 using separate buses. In this case, the machine-readable instructions 312 and data 340 may be stored in separate memory spaces, thereby implementing a Harvard architecture. Alternatively, the processor 302 may include one or more layers of cache, thereby implementing a modified Harvard architecture using only the one system bus 306. In some embodiments, the machine-readable instructions 312 are stored as an application in secondary storage (e.g., a hard drive), and loaded into the memory 308 upon powering on (i.e., boot up). In this case, the application and the data 340 share the same memory space, thereby implementing a von Neumann architecture.

### Applications

The benefits of the present embodiments stem from (1) the extremely high sensitivity of CE-DFCS, as compared to other types of spectroscopy, and (2) the ability of machine-learning techniques to quickly and efficiently model complex dependencies between variables and mechanisms that occur within the system and that give rise to the observed spectra. Accordingly, the present embodiments are particularly useful for applications where the sample (e.g., the gas 110 in FIG. 1) contains several atomic and/or molecular species whose concentrations depend on the states of the system in complex ways. This sections presents several such applications and examples. This section is not meant to be exhaustive, but rather representative of the wide range of systems and samples with which the present embodiments can work.

As an alternative to human breath, the sample may be another type of gas obtained from a human subject or a gas that is generated and collected by chemically processing a non-gas sample (i.e., solid or liquid) obtained from the human subject. Alternatively, the apparatus 100 may perform CE-DFCS directly on the non-gas (i.e., liquid or gas) sample. In these embodiments, the non-gas sample is placed within the cell 120 in lieu of the gas 110. Examples of non-gas liquid samples that may be obtained from the human subject and processed by the apparatus 100 include, but are not limited to, blood, saliva, urine, sweat, tears, and mucus. Examples of non-gas solid samples that may be obtained from the human subject and processed by the apparatus 100 include, but are not limited to, tissue samples (e.g., skin, muscle, fat, organ), stool samples, and placentae samples. Accordingly, the apparatus 100 may be used, for example, for blood analysis, urine analysis, autopsies, and the like.

SARS-CoV-2 can be detected and predicted by the present embodiments because its presence in the human body results in experimentally detectable changes in the concentrations of several molecular species in exhaled breath. Many other pathogens, diseases, and conditions can also produce experimentally detectable changes in concentrations (either in exhaled breath or another type of sample that can be obtained from the system) that the present embodiments can detect and use for prediction. Examples of human-based diseases and conditions that are known to affect the molecular makeup of breath include diabetes, pulmonology (e.g., asthma and chronic obstructive pulmonary disease (COPD)), oncology (e.g., lung cancer), neurodegenerative diseases (e.g., Parkinson's disease and Alzheimer's disease) and microbiome dysfunction.

For certain pathogens, diseases, and conditions, it remains unknown what, if any, effect they have on the concentrations of molecular species present in exhaled breath (or other detectable biomarkers in other types of sample). The present embodiments may be used as a tool to help identify such effects. If the effects result in experimentally detectable changes, the present embodiments may then be used to detect and predict the presence of such diseases and conditions. Accordingly, it should be understood that the present embodiments may be used to predict diseases and conditions whose biomarkers are still unknown.

In some embodiments, the system is an organism other than a human, such as a non-human animal. In these embodiments, the apparatus 100 may operate similarly to when the system is a human subject. For example, the sample may be breath exhaled, or other gas exerted, by the animal. Alternatively, the sample may be a non-gas liquid or solid sample obtained from the animal. These embodiments may be used, for example, for veterinary medicine, food safety, or as a tool for studying transmission of diseases both within and across different species.

In other embodiments, the system is, or includes, one or more microorganisms. For example, the sample may be water (or another fluid) containing a sample of bacteria. In these embodiments, metabolic processes of the microorganisms may change the composition of the fluid. Alternatively or additionally, these metabolic processes may produce gas (e.g., methane) that can be collected and used as the sample. Thus, in these embodiments the apparatus 100 may be used, for example, to monitor water safety or quantify a level of toxicity in the system. It should be understood from these examples, and others, that the system may be an entire ecosystem or a part thereof (e.g., a lake, geographical region, forest, section of a shoreline, etc.).

In other embodiments, the system is chemical. In these embodiments, the sample may be solid, liquid, or gas, regardless of the physical state of the system. In these embodiments, the apparatus 100 may be used, for example, at a chemical plant to monitor the presence or quantity of one or more certain chemicals (e.g., one or more intermediate products or one or more final products) that are produced during a sequence of one or more chemical processes. In this case, the model output 142 may be used to determine when to stop or alter a chemical process based on a quantity of an intermediate product. In one example, the apparatus 100 is used at a waste-water treatment facility and the model output 142 is a binary-value prediction indicating whether or not a sample passes a water quality standard. The model output 142 may additionally or alternatively indicate a quantity of a contaminant (e.g., an inorganic contaminant, a volatile organic contaminant, or a synthetic organic contaminant) detected in the sample.

In other embodiments, the system is mechanical, such as a machine. In these embodiments, the sample may be gas released by the machine as part of its operation. The apparatus 100 may analyze this gas, generating the model output 142 to indicate whether the engine is operating properly. For example, the system may be a vehicle with a combustion engine or an industrial furnace. In both cases, the sample may be exhaust. The concentrations of various molecular species in the exhaust (e.g., CO₂, CO, NO₂, SO₂, etc.) depends on the operating conditions of the system and the contents of the fuel used. The complex interdependencies of these variables can be quickly learned by the machine-learning model 144 and used to identify if the system is operating properly (e.g., the system is in a default “optimum” state). When the model output 142 indicates that the system is no longer in the “optimum” state, the apparatus 100 may control the system accordingly. For example, the apparatus 100 may shut down the engine or furnace such that a technician can investigate and perform any needed service or repairs. Alternatively or additionally, the apparatus 100 may perform diagnostic tests to gather more information about the system and its current state. Alternatively or additionally, the apparatus 100 may alter one or more parameters to return the system to a more-optimal operating state.

In other embodiments, the system is a manufacturing facility, such as a factory that manufactures a product according to a sequence of one or more production steps. In these embodiments, the apparatus 100 may be used, for example, to determine when a production step of the sequence has completed, and therefore when the sequence should continue to the next production step of the sequence. The apparatus 100 may then control the product line to stop the current production step, advance to the next production step, or both. In cases where the product is spectroscopically measurable, the apparatus 100 may also be used to test each product to determine if it passes specifications. Such testing may occur after any one or more of the production steps, or after the product is finished. Accordingly, the apparatus 100 may be used for quality control or quality assurance.

One application of the present embodiments is the manufacture of wine, liquors (e.g., whisky, scotch, brandy, rum, gin, tequila, vodka, etc.), and other types of distilled alcoholic beverages. Using wine as an example, the apparatus 100 may be used to analyze a sample of grape juice or must obtained from a vineyard (i.e., the system) to determine, based on the spectroscopic analysis, if the grapes are ready to harvest. The apparatus 100 may also be used to monitor the alcohol content in the must as it ferments, and therefore can identify when fermentation can end and bottling can begin. The apparatus 100 may further be used to monitor the wine during storage, tracking changes over time to its chemical composition, thereby allowing the vintner to, for example, better time its release to market.

Another application of the present embodiments related to wine and spirits is the detection of any number of various wine faults and defects. Examples of such wine faults include vinegar taint (i.e., presence of acetic acid), cork taint (i.e., presence of 2,4,6-trichloroanisole (TCA)), acetaldehyde, amyl-acetate, sulfur compounds (e.g., hydrogen sulfide and sulfur dioxide, mercaptans, etc.), iodine, lightstrike, and microbiological faults (e.g., geosmin, lactic acid bacteria, geranium taint, mousiness, refermentation, etc.). All of these wine faults produce distinct chemical changes in the wine that can be spectroscopically detected using CE-DFCS. Accordingly, the apparatus 100 may be used to detect one or more wine faults, in which case the system is a bottle of wine and the wine fault is a state of the system (e.g., the wine is “corked”). The apparatus 100 may automatically perform certain tasks when it classifies the bottle of wine as being in a “fault” state. For example, it may mark the bottle as faulty, dispose of the wine, notify a technician, or any combination thereof. The apparatus 100 may automatically perform other tasks when it classifies the bottle of wine as being in a non-fault state (i.e., a state without faults). For example, it may control a machine to pack the bottle in a box for shipment.

The present embodiments could potentially find use in various defense-related applications. One example is ultrasensitive, non-invasive, and non-destructive detection of volatile compounds (e.g., nitrogenated hydrocarbon groups, as in trinitro toluene (TNT)) for identifying unexploded explosives, ordnances, and munitions. Another example is detection of various molecular species to identify chemical and biological warfare agents.

Another application of the present embodiments related to wine and spirits is counterfeit detection. It is known that for certain types of spirits (e.g., scotch), different brands have different spectroscopic profiles. The apparatus 100 can be used to measure the spectroscopic profile of a sample of unknown origin. The machine-learning model 144 may be trained to compare this measured profile to known spectroscopic CE-DFCS profiles of various brands. If the apparatus 100 identifies a match (e.g., the output of the machine-learning model 144 is a probability exceeding a threshold), then a brand can be attributed to the sample. Alternatively, if the apparatus 100 does not find a match to any of the various brands, or finds a match that is different than what is claimed, then the apparatus 100 may conclude that the sample is counterfeit. In this case, the apparatus 100 may further perform one or more tasks, such as identifying a technician, printing a report, adding the measured spectrum to a database of spectra of known counterfeits, etc.

The present embodiments may also be used as a scientific tool, especially for understanding the reasons behind a particular prediction made by the machine-learning model 144. Predictions generated by the machine-learning model 144 can be very accurate if one or more detected molecular species show a sufficient change in concentration. For example, one cannot accurately predict whether a human subject was born in January or February just by measuring the molecular contents of their breath because no molecular species in exhaled breath has a concentration that varies with birth month. However, one can accurately predict whether a breath sample is exhaled air or inhaled air because the concentration of water molecules changes significantly. While some molecular species can be found in both exhaled air and inhaled air (e.g., methane), their concentration does not change much compared with water molecules, and therefore are less important for predicting whether a breath sample is exhaled or inhaled air.

It may be advantageous to understand how the change in concentrations of certain molecular species impact predictive accuracy. Such understanding provides insight into the workings of the system (e.g., the pathophysiology of diseases in medical-related applications). With this understanding, it may also be possible to construct a simplified or specialized device to detect only the important molecular species (i.e., those with high predictive power), which in turn can be used to achieve comparable prediction accuracy but possibly at a lower cost or overall suitability. Machine-learning processing of CE-DFCS spectra, as implemented by the present embodiments, can be used to identify which molecular species are the most important for predictive accuracy. This analytical capability allows one to uncover underlying scientific processes that cause the chemical compounds of different categories to differ.

Example algorithms for rating the importance of different molecular species include, but are not limited, to Variable Importance in Projection (VIP) score and comparisons of pattern-based and species-based approaches. As described in more detail below for the case of SARS-CoV-2 infection, the VIP score was used to identify H₂O, HDO, H₂CO, NH₃, CH₃OH, and NO₂ as the molecular species in exhaled breath that are the most important. By contrast, ¹²CH₄, ¹³CH₄, OCS, C₂H₄, CS₂, O₃, N₂O, SO₃, HCl, C₂H₆ are molecular species that are less important. With these results, the important molecular species can be studied further to further improve understanding of the underlying pathophysiology. For the example of SARS-CoV-2 infection, the pattern-based approach gives a higher prediction accuracy than the species-based approach, which indicates that additional unfitted molecular species are present and that these unfitted species may have predictive power. Follow-up studies can be pursued to try to uncover the identities of these unfitted species.

### Experimental Demonstration

### 1. Introduction

The difficulty to rapidly and accurately detect severe acute respiratory syndrome coronavirus 2 (SARS-CoV-2) infection has been a barrier to the response throughout the coronavirus disease 2019 (COVID-19) pandemic [1]. The current gold standard method, reverse transcription polymerase chain reaction (RT-PCR) test to detect viral RNA [2], requires appropriate sample collection and storage for accuracy, and is time-consuming [3]. Sampling is typically invasive (e.g., nasal swab), contributing to test hesitancy. The real-time assessment of community prevalence, implementation of public health protocols, and timely anti-viral intervention for high-risk people [4, 5], would all benefit significantly from the development of rapid, safe, sensitive, and non-invasive detection methods for SARS-CoV-2 infection, particularly with recent variants showing an increased epidemic growth rate [6].

Exhaled breath analysis is an attractive alternative to RT-PCR detection of SARS-CoV-2 infection as it is non-invasive and can return real-time measurements[7, 8]. Early studies to develop breath-based COVID-19 diagnosis included nanomaterial-based sensors[9, 10], ion-mobility spectrometry [11, 12], and mass spectrometry [13, 14]. A COVID-19 breath diagnostic test based on gas chromatography-mass spectrometry (GC-MS) was recently granted emergency use authorization by the U.S. Food and Drug Administration after its validation with over 2409 individuals, reporting 91.2% sensitivity and 99.3% specificity [15, 16]. While GC-MS currently represents one of the most powerful techniques for breath analysis due to its superior detection sensitivity and specificity [7, 17], breath molecules present with identical mass-to-charge ratio imposes real analytical challenges for mass spectrometry to discriminate. In addition, unavoidable alteration to breath components via purification, derivatization, and thermal degradation introduced from the use of a pre-concentrator [16] and a high-temperature thermal process [18] can also hinder accurate measurement of breath profiles.

The recently-developed laser spectroscopy-based technique of cavity-enhanced direct frequency comb spectroscopy (CE-DFCS) [19, 20] can help overcome the analytical challenges of mass spectrometry. CE-DFCS rapidly detects and identifies molecules in exhaled breath by ultra-sensitively measuring their structure-specific absorption signals via laser light at numerous optical frequencies. It requires no sample heating or purifying and ensures chemistry-free determinations of breath profiles. Together with the superior parts-per-trillion detection sensitivity [19], and with robust specificity to discriminate between different isomeric, isobaric, and isotopologue compounds[21], this technique offers rapid, accurate, and robust information that can add to diagnosis and mechanistic insight. Recent proof-of-principle studies have demonstrated the use of CE-DFCS to monitor changes in exhaled breath profiles upon fruit intake [19] and smoking [20], showing potential utility for disease diagnostics. To test if this powerful methodology may be useful for non-invasive medical diagnostics, a trial study was carried out for the first time to test its ability to identify SARS-CoV-2 infection in a young, highly, vaccinated cohort as a case study.

### 2. Method

### 2.1. Human Subjects

This study was approved by the Institutional Review Board (protocol no. 21-0088) of the University of Colorado Boulder. From May 2021 to January 2022, breath samples from a total of 170 research subjects were collected with a class distribution for SARS-CoV-2 infection of 83 positives (48.8%) and 87 negatives (51.2%). Research subjects were all University of Colorado Boulder affiliates, at least 18 years old, and recruited after taking a university-provided saliva-based or nasal swab COVID-19 RT-PCR test. The general campus population was >90% vaccinated. No participants were severely ill or requiring hospitalization at the time of their sample collection. After receiving their COVID-19 test results, potential subjects received a study recruitment email and were asked to contact the research team within 24 h if interested in participation. They then reviewed and signed an informed consent form, completed a questionnaire, and scheduled an appointment for the collection of their breath samples. The questionnaire collected self-reported information on sex, age, and race as well as other factors that could impact breath analysis including smoking, alcohol use, and underlying gastrointestinal symptoms. Additional information was collected on acute symptoms experienced by the positive participants. No viral genomes were sequenced, but the Colorado statewide data [22] over our subject recruitment period indicates infection with several viral variants associated with several infection waves (namely, alpha, delta, and omicron) in the community. All data (i.e., informed consent form, questionnaire, and Tedlar bag ID) were collected and managed using the REDCap electronic data capture tool [23, 24] hosted by the University of Colorado Denver.

### 2.2. Breath Sample Collection and Handling

Standard Tedlar bags (1 l, part no. 249-01-PP, SKC Inc.) were used to collect exhaled breath. During the sample collection appointment, research subjects were asked to hold their nose and breathe through their mouth. They were instructed to inhale to full lung capacity for 1-3 s, followed by exhaling the first half of their breath to the surroundings and the second half into the bag until the latter was above ˜80% full. The sample collection location was an outdoor university parking lot. The participants were not instructed to limit or control their smoking, food or alcohol intake prior to sample collection. Right after collection of one breath sample, the Tedlar bag was stored inside an air-tight container at ambient temperature and transported to the indoor lab housing the CE-DFCS setup for immediate data collection and analysis. The breath sample was warmed to 37° C. for 20 min to reduce condensation, then steadily flowed through the cleaned vacuum chamber held at room temperature (20° C.) at a rate of ˜1 l min⁻¹. Just before bag exhaustion, timely closure of the gas valves detained a portion of breath sample inside the chamber and a static pressure of 50 Torr (67 mbar) was reached (without re-condensation) for spectroscopic data collection. After the measurement, the breath sample was pumped out to an exhaust line leading to the building exterior. The used Ted-lar bag was autoclaved and disposed of. While direct sampling at atmospheric pressure by our breathalyzer is feasible, off-line sampling and negative pressure were adopted to ensure no SARS-CoV-2 could be introduced into the laboratory air. Spectroscopy data collection for each breath sample was completed in less than 10 min. This can be further reduced to about 1 s when optimized data acquisition and readout are implemented. Overall, from sample collection and transportation to completion of data analysis, the total time was less than an hour. Air samples were collected on separate days over the subject's recruitment period at the sample collection location as control specimens.

### 2.3. CE-DFCS Technique

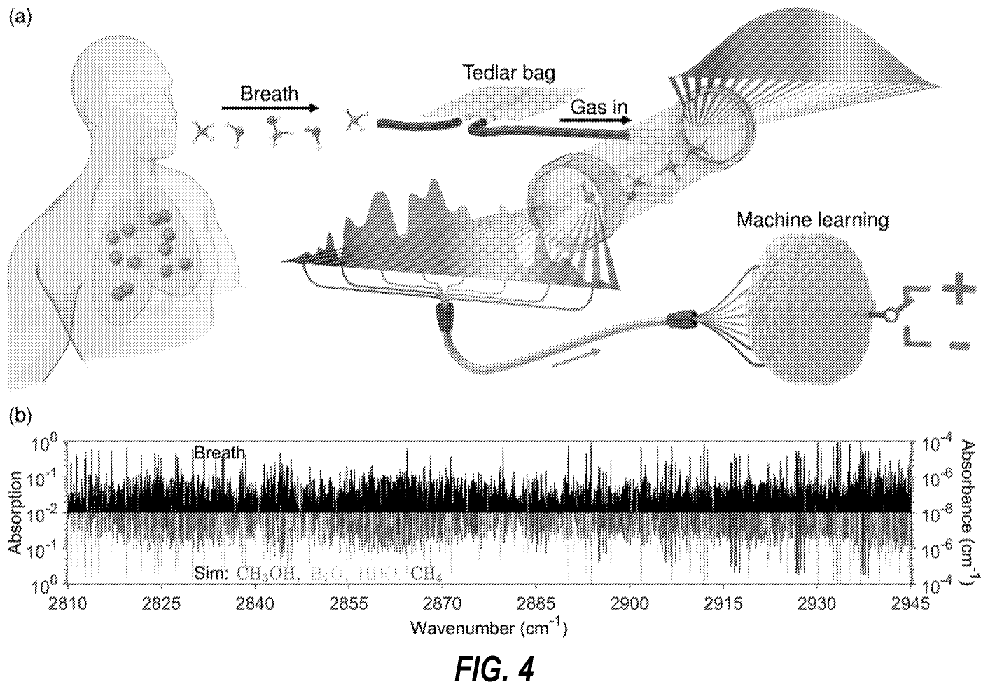

The working principle of the CE-DFCS breathalyzer is illustrated in panel (a) of FIG. 4. A high-resolution broadband absorption spectrum having a total of 14,836 distinct molecular features, each measured ultra-sensitively at individual optical frequencies, was recorded for each breath sample (see sample spectrum in panel (b) of FIG. 4). The breath spectrum was processed by machine learning analysis for binary response classifications. For additional instrument details, see [19].

### 2.4. Machine Learning Analysis

We employed two spectral pre-processing techniques for machine learning analysis: (1) a pattern-based approach that directly used all 14,836 molecular absorption features as the predictor variables; (2) a molecule-based approach that used 16 known small-molecule compounds (H₂O, HDO, ¹²CH₄, ¹³CH₄, OCS, C₂H₄, CS₂, H₂CO, NH₃, CH₃OH, O₃, N₂O, NO₂, SO₃, HCl, and C₂H₆) fitted to the spectra as predictor variables. The former approach identifies all stable patterns that can be used for diagnostics, whereas the latter identifies only the patterns that can be reduced to known molecular identities, which may result in loss of utilizable chemical information but allows better interpretability into the model details. The 16 compounds were chosen due to their availability from the high-resolution transmission molecular absorption database [25]. While more molecules can potentially be uncovered and fitted, quantitative extraction of their identities requires cross-sectional data at our experimental conditions (20° C. temperature and 50-Torr pressure) to be available. Unfitted species are hence not used in the molecule-based analysis despite being potentially useful to facilitate better predictive power.

To enable binary class assignment, we used partial least squares-discriminant analysis (PLS-DA) [26]. This method allows for the reduction of high-dimensionality data into a one-dimensional scalar number to differentiate between the opposing response classes (positive vs. negative). Variable importance in the projection (VIP) scores [27] were determined for assessing the relative importance of each predictor variable. To assess predictive power, the complete dataset (N=170) was randomly divided into a training set (n=140) and the remaining as a testing set (n=30). Both sets shared the same binary class distributions as the complete data set. The training set was used for model construction (a total of 15 PLS components were constructed) and the testing set was used for a blind test to obtain a receiver-operating-characteristic (ROC) curve, from which the area under the curve (AUC) value was calculated. Depending on how the complete set was divided, the AUC value obtained can vary to a certain extent. To ensure convergence, we repeated the whole process (i.e., cross-validation) for a total of 10,000 times, and each time a new training set and testing set were randomly re-selected for a new AUC value to be calculated. The ROC curves generated from the total of 10,000 cross-validation runs were averaged together to obtain an averaged ROC curve. The AUC of the averaged curve thus represents the average AUC from all cross-validation runs. To determine the AUC uncertainty, we used different training/testing partition ratios and different numbers of PLS components. All analysis code was written using MAT-LAB and the PLS-DA was performed using the built-in package based on the SIMPLS algorithm [28]. The supplementary file contains additional details on PLS-DA and VIP score principles, ROC averaging, and AUC uncertainties.

### 3. Results

### 3.1. Subject Characteristics

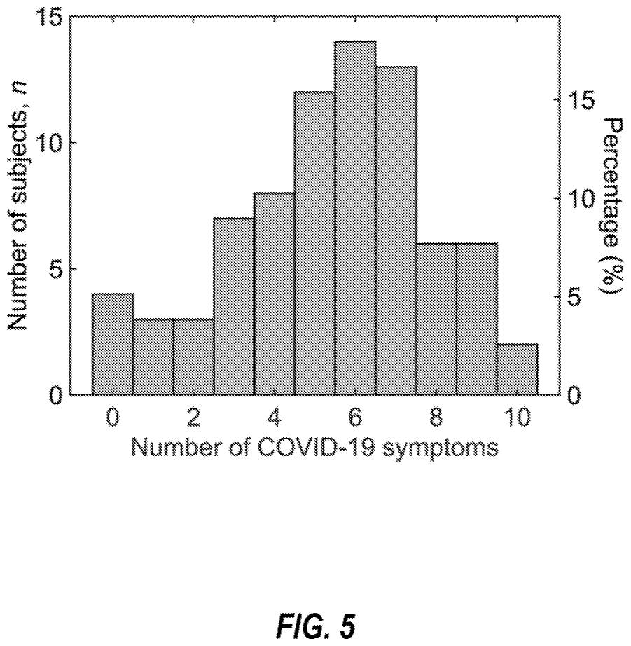

One-hundred and seventy participants enrolled in this study, with characteristics summarized below in Table 1. These included 83 (48.9%) SARS-CoV-2 positive subjects and 87 (51.2%) SARS-CoV-2 negative subjects based on prior RT-PCR tests. The median age was 22 years in the infection-positive and 24 years in the infection-negative groups (p<0.05). Both infection-positive and negative groups were balanced for sex (53.0% female infection-positives, 49.4% female negatives). Race and ethnicity distributions were equivalent between infection-positive and negative groups. A higher number of infection-negative subjects reported a history of rare to occasional abdominal symptoms, though there was no difference in the history of lactose intolerance or constipation between the two groups. SARS-CoV-2-positive subjects were asked additional questions regarding COVID-19-related symptoms, if any (See Table 2). We found most subjects reported multiple symptoms (see FIG. 5). Of 78 who responded, 50.0% reported 5-7 of the 11 listed symptoms, 5.1% were asymptomatic, and 2.6% reported 10 symptoms.

### 3.2. Comparable Prediction Accuracy for SARS-CoV-2 Infection by RT-PCR and CE-DFCS

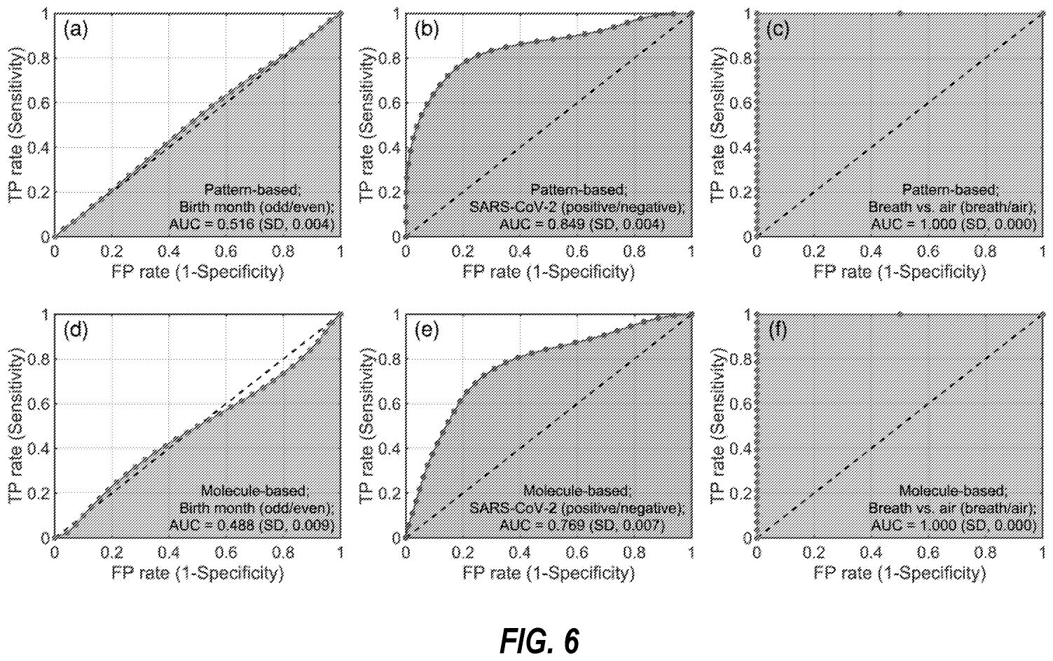

Breath analysis by laser spectroscopy can differentiate between SARS-CoV-2 infection positives and negatives. Using the two spectral pre-processing techniques for machine learning analysis, we found the pattern-based approach yielded an AUC of 0.849 (standard deviation [SD], 0.004) (see panel (b) in FIG. 6) and the molecule-based approach yielded an AUC of 0.769 (SD, 0.007) (see panel (e) in FIG. 6). Both approaches confirmed that significant differences in breath contents caused by SARS-CoV-2 infection was successfully detected by CE-DFCS. The classification results on SARS-CoV-2 infection should be interpreted as the co-agreement between the CE-DFCS breath test and the RT-PCR tests employed. As control experiments to validate the analysis methodology, we checked predictions for two cases with known responses: (1) a random guess based on subjects born in even vs. odd months, for which the lowest possible AUC of 0.5 is expected; (2) a perfect discrimination comparing ambient air vs. exhaled breath samples, for which one expects an AUC of 1. Both the pattern-based and molecule-based approaches confirmed expectations for results from a random sampling by birth month (see panels (a) and (d) in FIG. 6), yielding an AUC of 0.516 (SD, 0.004) and 0.488 (SD, 0.009) respectively. With regard to ambient air vs. breath, both approaches yielded AUCs of 1.000 (SD, 0.000) (see panels (c) and (f) in FIG. 6) and confirmed perfect discrimination criterion. These results further support the reliability of our analysis protocol. The AUC of ˜0.5 obtained from predictions of baseline response also suggested that our sample size was large enough to capture sufficient population diversity.

### 3.3. Pattern-Based Approach Outperforms Molecule-Based Approach

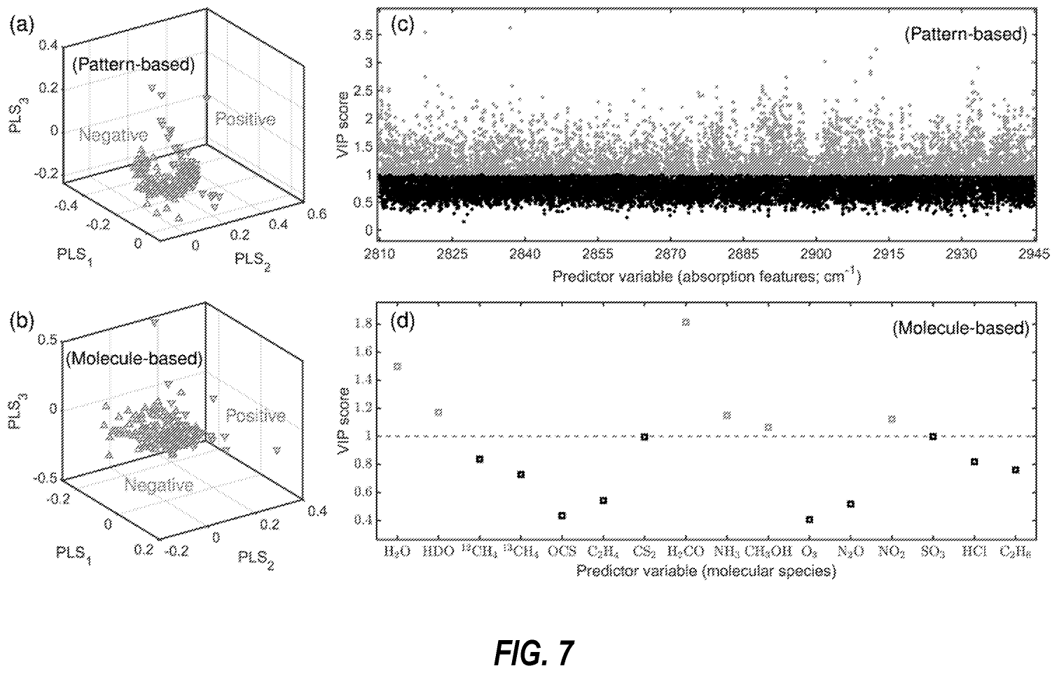

For SARS-CoV-2 infection, we found that the pattern-based approach clearly outperformed the molecule-based approach in prediction performance (AUC of 0.849 (SD, 0.004) vs. 0.769 (SD, 0.007)). To illustrate this result, we made use of the subjects' distribution on the PLS coordinate, which allowed us to visualize which approach can better discriminate opposing response classes. We used the complete data set (N=170) for construction of the PLS coordinate space and plotted subjects' data on the first three PLS components in panels (a) (pattern-based) and (b) (molecule-based) of FIG. 7. The results show significantly better discrimination capability was obtained by the pattern-based approach. The underperformance of the molecule-based approach could potentially be attributed to the exclusion of species with unknown identities in exhaled breath detected by CE-DFCS. As CE-DFCS acquires breath data at extremely high sensitivity, specificity, and dimensionality, applying the pattern-based approach to make full use of the wealth of chemical information collected by CE-DFCS is advantageous in that it bypasses the need for a complete molecular database to directly understand the best possible prediction power.

A notable limitation of the pattern-based approach, however, is that it does not reveal which molecules are important for making predictions, but only the optical frequencies at which they are probed. Variable importance analyzed for the pattern-based approach (see panel (c) of FIG. 7) identified prediction-important optical frequencies (VIP scores>1) where measured absorption values were strongly discriminative between SARS-CoV-2 positives and negatives. These frequencies are distributed near-uniformly over the entire spectrum. On the other hand, variable importance analyzed for the molecule-based approach (see panel (d) in FIG. 7) identified a panel of indicative molecular species for SARS-CoV-2 infection: water (H₂O), semiheavy water (HDO), formaldehyde (H₂CO), ammonia (NH₃), methanol (CH₃OH), and nitrogen dioxide (NO₂). Being able to identify the molecules provides better clarity to rationalize a possible prediction. To illustrate, variable importance performed for ambient air vs. breath samples based on the molecule-based approach identified water (H₂O) and semi heavy water (HDO) as the only important predictor variables (data not shown). This is easy to understand because water contents were saturated in breath and hence the machine could solely rely on them for prediction. The panel of indicative molecules identified by the molecule-based approach for SARS-CoV-2 infection provides the opportunity for further studies to elucidate the pathophysiology of SARS-CoV-2 infection.

TABLE 1
Participant characteristics.

| | | SARS-CoV-2 | SARS-CoV-2 | |
|---|---|---|---|---|
| | Total | positive | negative | |
| Characteristic | (N = 170) | (n = 83; 48.9%) | (n = 87; 51.2%) | P^{a} |
| Sex | | | | |
| Female | 87 (51.2) | 44 (53.0) | 43 (49.4) | 0.99 |
| Male | 83 (48.8) | 39 (47.0) | 44 (50.6) | |
| Age, median (IQR), years | 23 (8.8) | 22 (6) | 24 (10) | 0.01 |
| Race | | | | |
| Other/mix | 12 (7.0) | 4 (4.8) | 8 (9.2) | 0.53 |
| Asian | 20 (11.8) | 8 (9.6) | 12 (13.8) | |
| White | 138 (81.1) | 71 (85.5) | 67 (77.0) | |
| Latino | | | | |
| Yes | 14 (8.2) | 7 (8.4) | 7 (8.0) | 0.84 |
| No | 156 (91.8) | 76 (91.6) | 80 (92.0) | |
| Alcohol frequency, days week⁻¹ | | | | |
| d = 0 | 45 (26.5) | 15 (18.1) | 30 (34.5) | |
| 0 < d ≤ 3 | 114 (67.1) | 64 (77.1) | 50 (57.5) | 0.09 |
| 3 < d ≤ 7 | 11 (6.5) | 4 (4.8) | 7 (8.0) | |
| Smoker (Tobacco/Vape/Marijuana) | | | | |
| Yes | 31 (18.2) | 11 (13.3) | 20 (23.0) | 0.05 |
| No | 139 (81.8) | 72 (86.7) | 67 (77.0) | |
| Abdominal pain | | | | |
| Never | 79 (46.5) | 48 (57.8) | 31 (35.6) | 0.01 |
| Rarely | 50 (29.4) | 20 (24.1) | 30 (34.5) | |
| ≥Occasionally | 41 (24.1) | 15 (18.1) | 26 (29.9) | |
| Lactose intolerance | | | | |
| Not at all | 113 (66.5) | 60 (72.3) | 53 (60.9) | 0.15 |
| Very mild to mild | 34 (20.0) | 14 (16.9) | 20 (23.0) | |
| Moderate to severe | 23 (13.5) | 9 (10.8) | 14 (16.1) | |
| Constipation | | | | |
| Not at all | 140 (82.4) | 69 (83.1) | 71 (81.6) | 0.82 |
| Very mild | 19 (11.2) | 9 (10.8) | 10 (11.5) | |
| ≥Mild | 11 (6.4) | 5 (6.0) | 6 (6.9) | |

Information collected for the total of N = 170 participants (n = 83 positive; n = 87 negative).
Unless otherwise indicated, data are presented as n (%). IQR, interquartile range.
^{a}P values compare subjects positive and negative for SARS-CoV-2 infection.

TABLE 2
COVID-19 symptoms experienced by the positive participants.

| Characteristic^{a} (positive; N = 83) | No, n (%) | Yes, n (%) |
|---|---|---|
| Diarrhea | 67 (81.7) | 15 (18.3) |
| Fever or chills | 44 (53.7) | 38 (46.3) |
| Cough | 24 (29.3) | 58 (70.7) |
| Shortness of breath or difficult breathing | 64 (78.0) | 18 (22.0) |
| Fatigue | 22 (27.2) | 59 (72.8) |
| Muscle or body aches | 37 (45.1) | 45 (54.9) |
| Headache | 25 (30.5) | 57 (69.5) |
| New loss of taste or smell | 59 (72.8) | 22 (27.2) |
| Sore throat | 34 (42.0) | 47 (58.0) |
| Congestion or runny nose | 12 (14.6) | 70 (85.4) |
| Nausea or vomiting | 72 (88.9) | 9 (11.1) |

^{a}Information collected for the COVID-19 positive participants (N = 83) only. Statistics n (%) evaluated for those with non-missing values.

### 3.4. Prediction Performance for a List of Potential Confounders

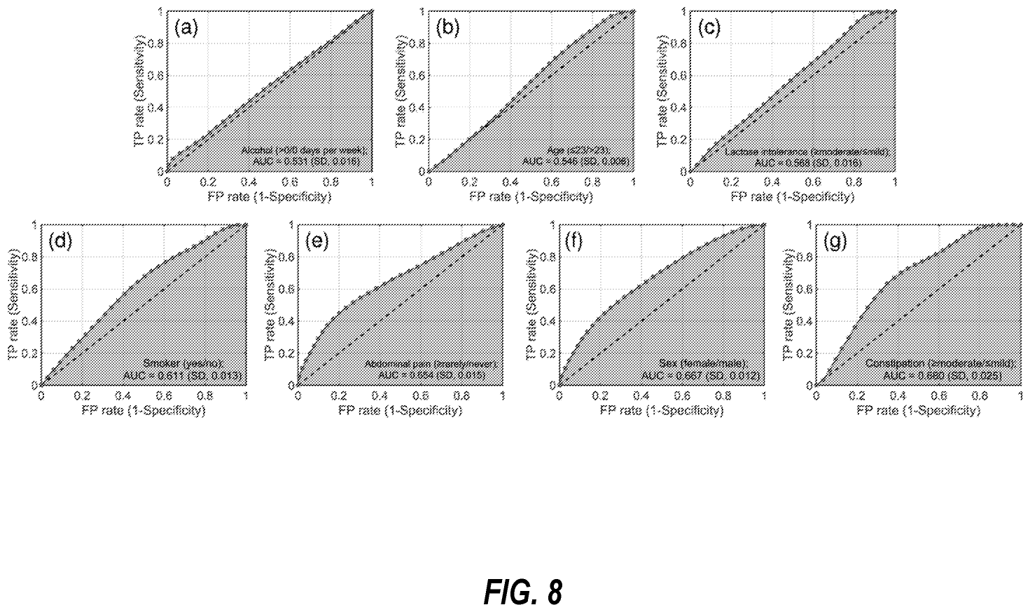

We analyzed the prediction performance for a list of subject characteristics and potential factors that could confound the results. For prediction of a specific response, subjects from the complete dataset (N=170) were divided into opposing classes based on the self-reported questionnaire data. Results obtained using the pattern-based approach are presented in FIG. 8 and the group assignment criteria for different response types are listed in the panels. A summary for all prediction analyses can also be found below in Table 3. From the results, we found random guessing predictions (AUC<0.6) for alcohol use, age, and lactose intolerance, but significant prediction capabilities for smoking, sex, abdominal pain, and constipation (0.6≤AUC<0.7). On age and abdominal pain, while our subjects had modest correlations with SARS-CoV-2 infection, the significantly better predictive power for SARS-CoV-2 infection suggests that age and abdominal pain do not constitute strong confounders. The superior prediction performance for SARS-CoV-2 infection compared to the list of potential confounders analyzed could potentially be due to SARS-CoV-2 infection eliciting acute and long-term host responses caused by both virus-driven and immune system-associated factors.

### 4. Discussion

We conducted the first pilot study to evaluate the diagnostic performance of CE-DFCS. Through a case study of SARS-CoV-2 infection detection involving 170 individuals, we found our pattern-based model produced excellent mutual agreement of 0.849 (SD, 0.004) AUC between the CE-DFCS test and the RT-PCR test results. Moreover, using the molecule-based model, we identified the relative importance of different breath molecules in making predictions. Finally, we present preliminary evidence that this technique could be extended to diagnose other conditions.

Our most important finding is that breath analysis by CE-DFCS can differentiate between SARS-CoV-2 infection positives and negatives. This study builds upon our prior works in which we established the use of CE-DFCS for the characterization of exhaled breath molecular profiles upon changes in biological conditions[19, 20]. Here, we have carried out the first trial study for CE-DFCS and employed machine learning analysis to realize robust binary diagnostics. Our study established CE-DFCS as a new diagnostic tool based on ultra-sensitive broadband laser spectroscopy. Continued assessment of CE-DFCS is important to thoroughly understand its diagnostic utility. Currently, the differences in the study designs make it difficult to compare the performance of CE-DFCS with GC-MS. The GC-MS study that has received FDA approval [15, 16] prospectively conducted RT-PCR tests and collected breath samples within 5 min of each other, restricted eating, drinking, or smoking for the 15 min preceding sample collection and excluded participation from those who had recent exposure to areas of local COVID-19 spread or close contact with COVID-19 positives. By contrast, our study had a much longer time delay from RT-PCR tests to breath sample collections (2.05 (SD, 0.95) days for the positives), and no exclusions based on travel/contact history. The time lag may result in viral clearance, and the more lenient sample collection and recruitment protocols may introduce confounders. These differences preclude a direct comparison of the two techniques. For future studies, examination of CE-DFCS's utility in individuals with severe disease or at higher risk, such as the elderly, the unvaccinated, and those with pre-disposing co-morbidities, will be important.

CE-DFCS may have broader applicability beyond the detection of SARS-CoV-2infection. It may also (1) serve as a non-invasive tool for evaluation of other health or biological conditions, and (2) provide insights into disease pathogenesis. With respect to (1), our results show that CE-DFCS discriminated between subjects based on smoking history [29, 30], biological sex [31-34], as well as gastrointestinal symptoms[35-37] (recurring abdominal pain and constipation). We were not able to discriminate subjects based on alcohol intake [38] or lactose intolerance [39], but this is not surprising as our subjects had not been specifically challenged with alcohol or lactose ingestion. With respect to (2), it has been recently reported [40] that SARS-CoV-2 virus exhibits strong optical absorption signals within our spectral coverage (2810 cm⁻¹-2945 cm⁻¹). This signal could potentially partly originate from the C—H molecular bonds in the surface-exposed SARS-CoV-2 spike protein [41]. A future measurement of the viral absorption spectrum in the gas phase with proper consideration of protein structure dynamics [42] may allow direct quantification of viral load in exhaled breath with CE-DFCS. This could allow us to examine the correlation between viral burden and other breath biomarkers and to determine the relative contributions of virus and host response to the change in breath molecular profiles. We find our results compelling enough to warrant future investigation into the applicability of CE-DFCS breath analysis to other conditions or diseases, particularly those of respiratory, gastrointestinal, or metabolic origin.

Finally, we note that ongoing rapid developments can further empower CE-DFCS in its use for medical diagnostics. Spectral range of the current CE-DFCS setup can be expanded to cover more ro-vibrational bands[43-46], thereby probing more discriminative features for stronger predictions. Furthermore, due to the direct measurement capability of CE-DFCS (i.e., no need for chemical treatments, pre-concentrations, and thermal processing), the technique can facilitate the creation of large-scale databases by accumulating breath data from different trial studies. This can promote the construction of deep learning model architectures[47-49] that can outperform traditional machine learning algorithms (e.g., PLS-DA) in predictive power. Recent photonics advances could potentially permit chip-scale miniaturization [50-52] for CE-DFCS and thus the technique could eventually be integrated into portable devices to support low-cost, widespread use and enable daily self-health monitoring on the go.

### 5. CONCLUSION

We present the first trial study of laser frequency comb spectroscopy for non-invasive medical diagnostics. Our case study of SARS-CoV-2 infection detection among a total of 170 individuals finds excellent mutual agreement between CE-DFCS and RT-PCR tests and supports the development of CE-DFCS as an alternative and accurate COVID-19 test with non-invasive sampling and rapid turnaround time. While the outstanding prediction performance was achieved using the pattern-based approach, continued enrichment in the molecular absorption database will empower high-resolution comb spectroscopy to employ molecule-based approach providing comparable prediction accuracy but with significantly better model interpretability. The laser spectroscopy-based technique, capable of ultra-sensitive, multi-species, rapid and chemistry-free detection of breath molecular contents with robust isomer-, isobaric-, and isotopologue-specificity opens a complementary approach for the development of breath-based diagnostics research.

### S1. Partial Least Squares-Discriminant Analysis (PLS-DA)

The principle of PLS regression and its usage for discriminant analysis, namely the PLS-DA algorithm, is briefly introduced here. The PLS regression toolbox used in our work was developed by MATLAB and implemented using the SIMPLS formulation. We discuss only the univariate response classification, corresponding to what is used in this work, but interested readers may consult Ref. [28] for more details beyond this classification type and how the actual algorithm is implemented. We use bold upper case to denote matrices, bold lower case for vectors, and un-bold for scalars, with primes (′) denoting a matrix or vector transpose. Collected data used for the training process are represented by the n×p predictor variables matrix X₀ and the n×1 univariate response variable vector y₀. Here, n is the total number of research subjects, p is the total number of predictor variables. Both X₀ and y₀ are column-centered so that the covariance of different predictor variables with the response can be expressed by a p×1 column vector

$${s}_{0} = {X}_{0}^{′} {y}_{0} .$$

PLS regression relates X₀ and y₀ based on y₀=X₀b+e, where b is the p×1 coefficients estimate, X₀b is the explained component, and e is the fit residual. In contrast to least squares regression, where the coefficients estimate b is constructed by minimizing the residual sum of squares e′e, PLS regression constructs it based on the covariance

$${s}_{0} = {X}_{0}^{′} {y}_{0}$$

to get more stabilized values of b and achieve more reliable predictive power. The formulation begins by projecting the predictor variables matrix X₀ onto a new coordinate system T=X₀R of reduced dimensionality spanned by a total of A (≤p−1) PLS components, where R denotes the p×A weight transfer matrix and T denotes the n×A projected scores matrix. The construction of R is subject to two constraints: 1) the covariance vector T′y₀ is maximized for each entry, meaning each PLS component exhibits the largest possible covariance with the response; 2) the PLS components are orthonormal, i.e., columns of T satisfy

$${t}_{i}^{′} {t}_{j} = {δ}_{i j}$$

for any i,j=1,2, . . . , A, where δᵢⱼ is the Kronecker delta. The coefficients estimate b can be determined once R is known, since

$${y}_{0} = {TT}^{′} {y}_{0} = {X}_{0} {RR}^{′} {X}_{0}^{′} {y}_{0} = {X}_{0} b ,$$

and thus

$$b = {RR}^{′} {X}_{0}^{′} {y}_{0} = {RR}^{′} {s}_{0} .$$

The process of determining R proceeds column by column. For the first iteration step k=1, the maximization of the covariance of the first PLS component (tₖ=X₀rₖ) with the response,

$${t}_{k}^{′} {y}_{0} = {r}_{k}^{′} {X}_{0}^{′} {y}_{0} = {r}_{k}^{′} {s}_{0} = max ,$$

constrains the first weight vector r_{k (k=}1) to be along the direction of s₀. For steps k>1, the orthogonany condition

$${t}_{k}^{′} {t}_{i} = {r}_{k}^{′} ( {X}_{0}^{′} {t}_{i} ) = 0$$

for i=1,2, . . . , k−1, requires the newly constructed rₖ to be orthogonal to each of the p×1 vectors

$${X}_{0}^{′} {t}_{i} for i = 1 , 2 , … , k - 1 .$$

We define

$${p}_{i} ≡ {X}_{0}^{′} {t}_{i}$$

as the loading vectors. One may use the Gram-Schmidt process to find the orthonormal basis of the subspace Vₖ₋₁ spanned by the loading vectors Pᵢ (i=1,2, . . . , k−1) and then determine the p×p projection operator P^{⊥} for the orthogonal complement space

$${V}_{k - 1}^{⊥} .$$

This loosely constrains the direction of rₖ to be within

$${V}_{k - 1}^{⊥} ,$$

requiring rₖ=P^{⊥}rₖ. Now, with the covariance maximization criteria,

$${t}_{k}^{′} {y}_{0} = {r}_{k}^{′} ( {P}^{{⊥}^{′}} {s}_{0} ) = max ,$$

the direction of rₖ is ultimately determined to be along the direction of the vector P^{⊥}′s₀, which is the projection of the covariance vector so onto the subspace

$${V}_{k - 1}^{⊥} .$$

The iteration process proceeds until the directions of all rₖ are determined, where the normalization condition T′T=1 governs the magnitudes of rₖ. Finally, the coefficients estimate is determined and can be used for prediction of the response class for new observations based on

$${y}_{0}^{pred} = {X}_{0}^{n e w} b ,$$

where the m×p matrix

$${X}_{0}^{n e w}$$

is the testing data for a total of m research subjects. The m×1 predicted values

$${y}_{0}^{p r e a}$$

are translated proportionally into posterior probabilities and compared with a threshold value for response class assignment.

### S2. Variable Importance in the Projection (VIP) Scores

In PLS-DA, assessment of the importance of the predictor variables needs to consider 1) the weighting of a given predictor variable to form different PLS components and 2) the importance of different PLS components in explaining the response. Regarding 1), the formation of the a^{th} PLS component (a=1, 2, . . . , A) takes the contribution from the j^{th} predictor variable with the normalized weight given by wⱼₐ/∥wₐ∥, where wⱼₐ is the j^{th} row a^{th} column element from the p×A weight matrix R, and

$$‖ {w}_{a} ‖ = {( {Σ}_{j = 1}^{p} {w}_{j a}^{2} )}^{1 / 2}$$

is the normalization. Regarding 2), we first note that the variance of the response among all observations

$${y}_{0}^{′} {y}_{0}$$

explained by the total of A PLS components to the extent of

$${\hat{y}}_{0}^{′} {\hat{y}}_{0} ,$$

where ŷ₀=X₀b=y₀−e. The total percentage variance explained in the response,

$$( {\hat{y}}_{0}^{′} {\hat{y}}_{0} / {y}_{0}^{′} {y}_{0} ) \times 100 \% ,$$

can be used for estimating the minimum number of PLS components needed for reliable predictions. The explained variance

$${y}_{0}^{′} {y}_{0} = {y}_{0}^{′} {TT}^{′ {y}_{0}} = {Σ}_{a = 1}^{A} {( {\hat{y}}_{0}^{′} {t}_{a} )}^{2}$$

is further broken down into a summation of the square of the covariance of all PLS components with ŷ₀. We can thus evaluate the importance of the a^{th} PLS component by its variance explained

$${q}_{a}^{2} ≡ {( {\hat{y}}_{0}^{′} {t}_{a} )}^{2} ,$$

a quantity assigning larger importance to the PLS components that have larger covariance with the explained component, with the total variance explained by the A PLS components given by

$${Σ}_{a = 1}^{A} {q}_{a}^{2} .$$

Taking both 1) and 2) into account, the variable importance for the predictor variable j summing over all the A PLS components is proportional to

$${[ {Σ}_{a = 1}^{A} {q}_{a}^{2} \cdot {( {w}_{j a} / ‖ {w}_{a} ‖ )}^{2} ]}^{1 / 2} .$$

From this, one can define its VIP score [27], a metric for characterizing its importance, by

$$VIP = \sqrt{\frac{p \cdot {Σ}_{a = 1}^{A} [ {q}_{a}^{2} \cdot {( {w}_{j a} / ‖ {w}_{a} ‖ )}^{2} ]}{{Σ}_{a = 1}^{A} ( {q}_{a}^{2} )}} . \qquad ( 1 )$$

Normalization ensures the mean square sums of the VIP scores among all predictor variables equals unity,

$${p}^{- 1} {Σ}_{j - 1}^{p} V I {P}_{j}^{2} = 1 .$$

Because of this normalization, predictor variables with VIP scores above (or below) unity can be regarded as important (or unimportant) variables.

### S3. Variance Explained by the PLS Components

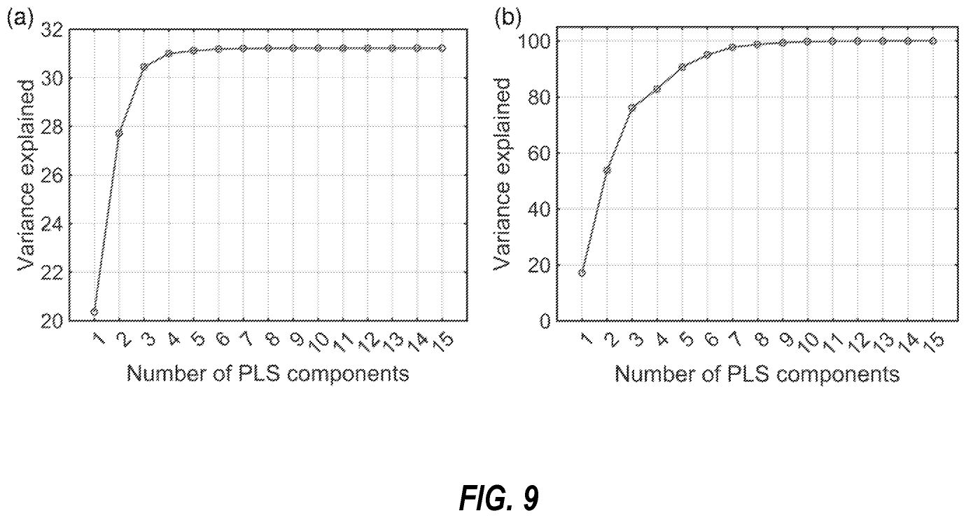

For SARS-CoV-2 infection classification, the total percentage variance explained in the response analyzed by the molecule-based and the pattern-based approaches for the complete data set (N=170) are given in FIG. 9. We found a sharp rise in the variance explained for both the molecule-based and the pattern-based approaches when the number of PLS components constructed lies in the range from unity to five. A total of 15 PLS components were sufficient to saturate the percentage variance explained for both approaches. The lower variance explained obtained by the molecular species-based approach suggests fitting the spectroscopy data with more molecular species can better explain the response.

### S4. Averaging of the Receiver-Operating-Characteristics Curves

We performed averaging of the ROC curves using the non-parametric method

adapted from Ref. [53]. This method ensured that: 1) the AUC of the averaged curve equaled the average AUC of individual cross-validation runs, and 2) the averaged AUC for a perfect (or random) classifier was equal to 1 (or 0.5). Proof for statement 1) can be found in the appendix of Ref. [53], while statement 2) can be straightforwardly deduced from 1). In our work, we averaged the individual ROC curves vertically in the tilted space formed by rotating the (FP,TP) axes counter-clockwise by an angle θ<π/2, where FP and TP denotes false positive rates and true positive rates, respectively. This enabled the averaging to be taken over singular functions. Any data point from an individual ROC curve could take its FP values from {(0, 1, 2, . . . , N)/N}, and TP values from {(0, 1, 2, . . . , P)/P}. Since we were using stratified sampling at the fixed testing set size Lₜₑₛₜ=P+N, different cross-validation runs preserved the total number of positives P and negatives N. Hence, we chose θ=arctan (P/N) such that the curve averaging in the tilted space would be performed to yield a total of (Lₜₑₛₜ+1) sample points for plotting the averaged ROC curve. The j^{th} (j=0, 1, 2, . . . , Lₜₑₛₜ) sample point represented the j^{th} observation in the testing set scanned over by the threshold line, and was obtained from the statistical mean over a total of the number of cross-validation runs of the j^{th} observation from each run.

### S5. Uncertainty in the AUC

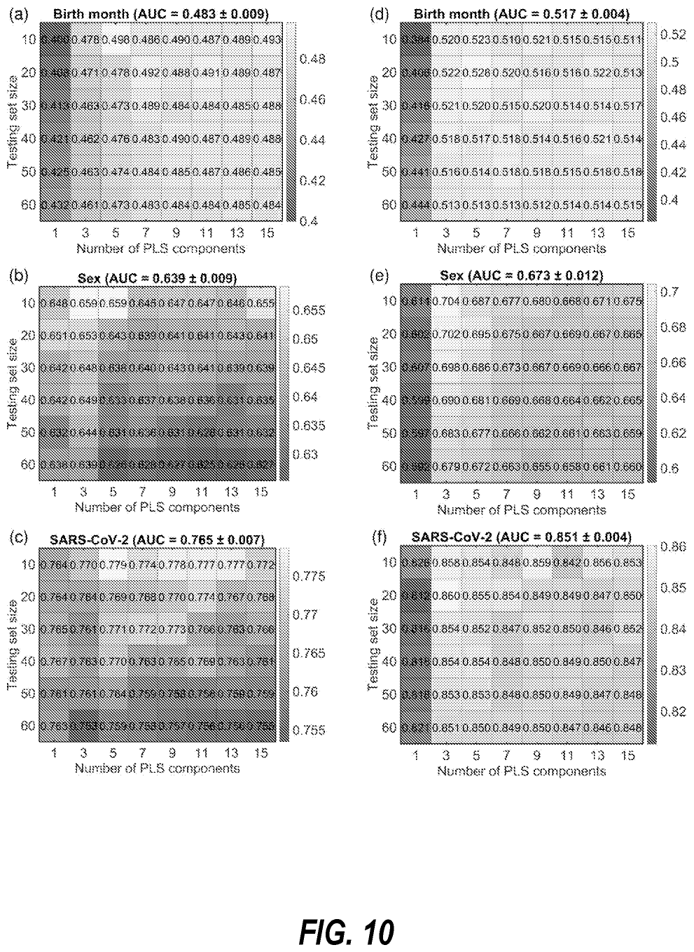

Uncertainty in the AUC for different response types was calculated using different numbers of PLS components and different partition ratios of the training and testing set (see FIG. 10). For each number of PLS components and partition ratio used, an AUC value was calculated from the averaged ROC curve obtained from 1,000 cross-validation runs based on stratified random sampling. As seen in FIG. 10, the AUC values calculated with only one PLS component were found to give worse prediction performance in general for both the molecule-based and the pattern-based approaches. This is understandable because both approaches showed limited total percentage variance explained when only one PLS component was constructed (see FIG. 10). For this reason, we calculated the mean and standard deviation of the AUC for each plot excluding those obtained using only one PLS component. Obtained values are reported in the title of each plot. The standard deviations were used as the uncertainty of AUC. The means were provided for reference. Note that in the main text the absolute values quoted for the AUC were computed using 15 PLS components, 140:30 training and testing partition ratio, and 10,000 cross-validation runs. We found the computed values using these settings matched the means obtained here to within the calculated uncertainty.

### S6. Prediction Performance Summary

A summary of binary response classification results for various response types is provided in Table 3. The obtained AUC shown for each response type were the mean and standard deviation calculated for the results obtained using 1,000 cross-validation runs based on stratified random sampling, evaluated at 3, 5, 7, . . . , 15 PLS components, and at 10, 20, 30, . . . , 60 test set size with training set size given by subtracting the testing set size from the complete data set.

TABLE 3
Prediction performance summary.

| | | Positive/Negative | Obtained | |
|---|---|---|---|---|
| | Positive/Negative | class distributions, | AUC, | Discrimination |
| Response | class assignment | n (%) | mean, (SD) | capability |
| Birth day | Odd/Even | 83 (48.8)/87 (51.2) | 0.510 (21) | Random |
| | | | | guessing |
| Birth month | Odd/Even | 83 (48.8)/87 (51.2) | 0.517 (4) | Random |
| | | | | guessing |
| Alcohol | >0 days per week/ | 125 (73.5)/45 (26.5) | 0.542 (16) | Random |
| frequency | 0 days per week | | | guessing |
| Age | Below 23 yr | 87 (52.1)/80 (47.9) | 0.549 (6) | Random |
| | (median)/ | | | guessing |
| | above median | | | |
| Lactose | Moderate to | 23 (13.5)/147 (86.5) | 0.574 (16) | Random |
| intolerance | very severe/ | | | guessing |
| | Not at all to mild | | | |
| Smoker | Yes/No | 31 (18.2)/139 (81.8) | 0.604 (13) | Significant |
| Abdominal | Rarely to frequently/ | 91 (53.5)/79 (46.4) | 0.660 (15) | Significant |
| pain | Never | | | |
| Sex | Female/Male | 87 (51.2)/83 (48.8) | 0.673 (12) | Significant |
| Constipation | Moderate to | 11 (6.5)/159 (93.5) | 0.674 (25) | Significant |
| | very severe/ | | | |
| | Never to mild | | | |
| SARS-CoV-2 | Infected/ | 83 (43.8)/87 (51.2) | 0.851(4) | Excellent |
| | Not infected | | | |
| Breath or Air | Breath/Air | 170 (91.9)/15 (8.1) | 1.000(0) | Perfect |

### REFERENCES

[1] Ritchie H et al 2020 Coronavirus Pandemic (COVID-19) Our World in Data
[2] Centers for Disease Control and Prevention 2020 Interim guidelines for collecting, handling, and testing clinical specimens for COVID-19 (available at: www.cdc.gov/coronavirus/2019-ncov/lab/guidelines-clinical-specimens.html)
[3] Mei X et al 2020 Artificial intelligence-enabled rapid diagnosis of patients with COVID-19 Nat. Med. 26 1224-8
[4] Larremore D B, Wilder B, Lester E, Shehata S, Burke J M, Hay J A, Tambe M, Mina M J and Parker R 2021 Test sensitivity is secondary to frequency and turnaround time for COVID-19 screening Sci. Adv. 7 eabd5393
[5] Saravolatz L D, Depcinski Sand Sharma M 2022 Molnupiravir and Nirmatrelvir-Ritonavir: oral COVID antiviral drugs Clin. Infect. Dis. 76 165-71
[6] Backer J A et al 2022 Shorter serial intervals in SARS-CoV-2 cases with Omicron BA. 1 variant compared with Delta variant, the Netherlands, 13 to 26 Dec. 2021Eurosurveillance 27 2200042
[7] Wang C and Sahay P 2009 Breath analysis using laser spectroscopic techniques: breath biomarkers, spectral fingerprints and detection limits Sensors 9 8230-62
[8] Arnold C 2022 Diagnostics to take your breath away Nat. Biotechnol. 40 990-3
[9] Shan B et al 2020 Multiplexed nanomaterial-based sensor array for detection of COVID-19 in exhaled breath ACS Nano 14 12125-32
[10] Zamora-Mendoza B N, de León-Martínez L D, Rodríguez-Aguilar M, Mizaikoff B and Flores-Ramírez R 2022 Chemometric analysis of the global pattern of volatile organic compounds in the exhaled breath of patients with COVID-19, post-COVID and healthy subjects. Proof of concept for post-COVID assessment Talanta 236 122832
[11] Ruszkiewicz D M et al 2020 Diagnosis of COVID-19 by analysis of breath with gas chromatography-ion mobility spectrometry—a feasibility study eClinicalMedicine 29-30 100609
[12] Chen H et al 2021 COVID-19 screening using breath-borne volatile organic compounds J. Breath Res. 15 047104
[13] Ibrahim W et al 2021 Diagnosis of COVID-19 by exhaled breath analysis using gas chromatography-mass spectrometry ERJ Open Res. 7 3
[14] Grassin-Delyle S et al 2021 Metabolomics of exhaled breath in critically ill COVID-19patients: a pilot study eBioMedicine 63 103154
[15] U.S. Food & Drug Administration Coronavirus (COVID-19) Update: FDA Authorizes First COVID-19 Diagnostic Test Using Breath Samples 2022 (available at: www.fda.gov/news-events/press-announcements/coronavirus-covid-19-update-fda-authorizes-first-covid-19-diagnostic-test-using-breath-samples)
[16] U.S. Food & Drug Administration 2022 InspectIR COVID-19 Breathalyzer (for use on PNY-1000) (available at: https://fda.report/media/157723/EUA-InspectIR-Breath-ifu.pdf)

[17] Smith D, Španěl P, Herbig J and Beauchamp J 2014 Mass spectrometry for real-time quantitative breath analysis J. Breath Res. 8 027101
[18] Fang M, Ivanisevic J, Benton H P, Johnson C H, Patti G J, Hoang L T, Uritboonthai W, Kurczy M E and Siuzdak G 2015 Thermal degradation of small molecules: a global metabolomic investigation Anal. Chem. 87 10935-41
[19] Liang Q, Chan Y-C, Changala P B, Nesbitt D J, Ye J and Toscano J 2021 Ultrasensitive multispecies spectroscopic breath analysis for real-time health monitoring and diagnostics Proc. Natl Acad. Sci. 118 e2105063118
[20] Thorpe M J, Balslev-Clausen D, Kirchner M S and Ye J 2008 Cavity-enhanced optical frequency comb spectroscopy: application to human breath analysis Opt. Express 16 2387-97
[21] Kranenburg R F, Peroni D, Affourtit S, Westerhuis J A, Smilde A K and van Asten A C 2020 Revealing hidden information in GC-MS spectra from isomeric drugs: chemometrics based identification from 15 eV and 70 eV EI mass spectra Forensic Chem. 18 100225
[22] Colorado Department of Public Health & Environment 2022 COVID-19 Variant Sentinel Surveillance (available at: https://covid19.colorado.gov/data)
[23] Harris P A, Taylor R, Thielke R, Payne J, Gonzalez N and Conde. J G 2009 Research electronic data capture (REDCap)—A metadata-driven methodology and workflow process for providing translational research informatics support J. Biomed. Inform. 42 377-81
[24] Harris P A et al 2019 The REDCap consortium: building an international community of software platform partners J. Biomed. Inform. 95 103208
[25] Gordon I E et al 2017 The HITRAN2016 molecular spectroscopic database J. Quant. Spectrosc. Radiat. Transfer 203 3-69
[26] Lee L C, Liong C-Y and Jemain A A 2018 Partial least squares-discriminant analysis (PLS-DA) for classification of high-dimensional (HD) data: a review of contemporary practice strategies and knowledge gaps Analyst 143 3526-39
[27] Chong I-G and Jun C-H 2005 Performance of some variable selection methods when multicollinearity is present Chemometr. Intell. Lab. Syst. 78 103-12
[28] de Jong S 1993 SIMPLS: an alternative approach to partial least squares regression Chemometr. Intell. Lab. Syst. 18 251-63
[29] Kushch I et al 2008 Compounds enhanced in a mass spectrometric profile of smokers' exhaled breath versus non-smokers as determined in a pilot study using PTR-MS J. Breath Res. 2 026002
[30] Buszewski B, Ulanowska A, Ligor T, Denderz N and Amann A 2009 Analysis of exhaled breath from smokers, passive smokers and non-smokers by solid-phase microextraction gas chromatography/mass spectrometry Biomed. Chromatogr. 23 551-6
[31] Grasemann H, van's Gravesande K S, Büscher R, Drazen J M and Ratjen F 2003 Effects of sex and of gene variants in constitutive nitric oxide synthases on exhaled nitric oxide Am. J. Respir. Crit. Care Med. 167 1113-6
[32] Taylor D et al 2007 Factors affecting exhaled nitric oxide measurements: the effect of sex Respir. Res. 8 82
[33] Good N et al 2021 Respiratory aerosol emissions from vocalization: age and sex differences are explained by volume and exhaled CO₂ Environ. Sci. Technol. Lett. 8 12
[34] Antufjew H M, A, Antufjew A, Borgmann K, Hempel F, Ittermann T, Welzel S, Weltmann K D, Völzke H and Röpcke J 2011 Influence of age and sex in exhaled breath samples investigated by means of infrared laser absorption spectroscopy J. Breath Res. 5 027101
[35] Pichetshote N and Rezaie A 2018 Breath tests for functional gastrointestinal disorders: when and for what? NeuroGastroLATAM Rev. 2 87-97
[36] De Lacy Costello B, Ledochowski M and Ratcliffe N 2013 The importance of methane breath testing: a review J. Breath Res. 7 024001
[37] Dutta Banik G, De A, Som S, Jana S, Daschakraborty S B, Chaudhuri Sand Pradhan M 2016 Hydrogen sulphide in exhaled breath: a potential biomarker for small intestinal bacterial overgrowth in IBS J. Breath Res. 10 026010
[38] Hlastala M 1998 The alcohol breath test-a review J. Appl. Physiol. 84 401-8
[39] Di Costanzo M and Berni Canani R 2019 Lactose intolerance: common misunderstandings Ann. Nutrition Metab. 73 30-37
[40] Barauna V G, Singh M N, Barbosa L L, Marcarini W D, Vassallo P F, Mill J G, Ribeiro-Rodrigues R, Campos L C G, Warnke P H and Martin F L 2021 Ultrarapid on-site detection of SARS-CoV-2 infection using simple ATR-FTIR spectroscopy and an analysis algorithm: high sensitivity and specificity Anal. Chem. 93 2950-8
[41] Soares J et al 2021 Diagnostics of SARS-CoV-2 infection using electrical impedance spectroscopy with an immunosensor to detect the spike protein Talanta 239 123076
[42] López-Lorente A Iand Mizaikoff B 2016 Mid-infrared spectroscopy for protein analysis: potential and challenges Anal. Bioanal. Chem. 408 2875-89
[43] Iwakuni K, Porat G, Bui T Q, Bjork B J, Schoun S B, Heckl O H, Fermann M E and Ye J 2018 Phase-stabilized 100 mW frequency comb near 10 μm Appl. Phys. B 124 1289
[44] Scalari G, Faist J and Picqué N 2019 On-chip mid-infrared and THz frequency combs for spectroscopy Appl. Phys. Lett. 114 150401
[45] Guo H, Weng W, Liu J, Yang F, Hänsel W, Brès C S, Thévenaz L, Holzwarth R and Kippenberg T J 2020 Nanophotonic supercontinuum-based mid-infrared dual-comb spectroscopy Optica 7 1181-8
[46] Lesko D, Timmers H, Xing S, Kowligy A, Lind A J and Diddams S A 2021 A six-octave optical frequency comb from a scalable few-cycle erbium fibre laser Nat. Photon. 15 281-6
[47] Amato F, López A, Peña-Méndez E M, Vaňhara P, Hampl A and Havel J 2013 Artificial neural networks in medical diagnosis J. Appl. Biomed. 11 47-58
[48] Al Ibrahim E and Farooq A 2021 Prediction of the derived cetane number and carbon/hydrogen ratio from infrared spectroscopic data Energy and Fuels 35 8141-52
[49] Enders A A, North N M, Fensore C M, Velez-Alvarez J and Allen H C 2021 Functional group identification for FTIR spectra using image-based machine learning models Anal. Chem. 93 9711-8
[50] Xiang C et al 2021 Laser soliton microcombs heterogeneously integrated on silicon Science 373 99-103
[51] Jin N, McLemore C A, Mason D, Hendrie J P, Luo Y, Kelleher M L, Kharel P, Quinlan F, Diddams S A and Rakich P T 2022 Micro-fabricated mirrors with finesse exceeding one million Optica 9 965-70
[52] Fathy A, Sabry Y M, Nazeer S, Bourouina T and Khalil D A 2020 On-chip parallel Fourier transform spectrometer for broadband selective infrared spectral sensing Microsyst. Nanoeng. 6 1-9.
[53] Chen, W and Samuelson F W 2014 The average receiver operating characteristic curve in multireader multicase imaging studies Brit. J. Radiol. 87 20140016.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method for analyzing a system, comprising:
performing cavity-enhanced direct frequency-comb spectroscopy to obtain an absorption spectrum indicating transmission of an optical frequency comb through a sample derived from the system; and
feeding the absorption spectrum into a machine-learning model to generate a model output, the machine-learning model having been trained with a supervisory set of cavity-enhanced direct frequency-comb spectra.

2. The method of claim 1, further comprising outputting the model output.

3. The method of claim 1, wherein:
the machine-learning model was trained with the supervisory set to classify each of the cavity-enhanced direct frequency-comb spectra into one of a plurality of states of the system; and
the model output includes a prediction that is one of the plurality of states.

4. The method of claim 3, each of the plurality of states being a disease state, a non-disease state, a physiological state, a chemical state, a medical state, or a functional state.

5. The method of claim 3, at least one of the plurality of states indicating the presence of an infection caused by a pathogen in the system.

6. The method of claim 5, the pathogen comprising the SARS-CoV-2 virus.

7. The method of claim 1, wherein:
the machine-learning model was trained with the supervisory set to perform regression on each of the cavity-enhanced direct frequency-comb spectra; and
the model output includes a test score indicating a severity of a state of the system.

8. The method of claim 7, the state being a disease state, a non-disease state, a physiological state, a chemical state, a medical state, or a functional state.

9. The method of claim 7, the test score indicating severity of an infection caused by a pathogen in the system.

10. The method of claim 9, the pathogen comprising the SARS-CoV-2 virus.

11. The method of claim 1, wherein the system is a human subject.

12. The method of claim 11, wherein the sample is a breath sample obtained from the human subject.

13. The method of claim 11, further comprising diagnosing, based on the model output, the human subject with a disease.

14. The method of claim 13, further comprising providing the human subject with a therapeutic intervention for treating the disease.

15. The method of claim 14, the therapeutic intervention comprising one or more of a surgical procedure, a non-surgical medical procedure, and a prescription for one or more pharmaceutical drugs.

16. The method of claim 1, wherein:
the absorption spectrum comprises a plurality of data points, each of the plurality of data points indicating transmission of a respective one of a plurality of comb teeth of the optical frequency comb through the sample; and
said feeding comprises feeding each of the plurality of data points into a respective one of a plurality of input nodes of the machine-learning model.

17. The method of claim 1, wherein:
the method further comprises generating a plurality of measured concentrations of a plurality of chemical constituents in the sample by fitting at least part of the absorption spectrum to each of a plurality of simulated absorption spectra corresponding to the plurality of chemical constituents; and
said feeding comprises feeding the plurality of measured concentrations into the machine-learning model.

18. An apparatus for analyzing a system, comprising:
a memory storing a machine-learning model that was trained with a supervisory set of cavity-enhanced direct frequency-comb spectra; and
a signal processor in electronic communication with the memory, the signal processor being configured to:
receive an absorption spectrum obtained from a cavity-enhanced direct frequency-comb spectrometer, the absorption spectrum indicating transmission of an optical frequency comb through a sample derived from the system; and
feed the absorption spectrum into the machine-learning model to generate a model output.

19. The apparatus of claim 18, further comprising the cavity-enhanced direct frequency-comb spectrometer.

20. The apparatus of claim 18, the signal processor being configured to output the model output.
